(19) United States
(12) Patent Application Publication
(10) Pub. No.: US 2025/0302552 A1
(43) Pub. Date: Oct. 2, 2025

(54) INTERFACE FOR IDENTIFYING OBJECTS IN AN ANATOMY

(71) Applicant: AURIS HEALTH, INC., Santa Clara, CA (US)

(72) Inventors: Jeffrey Adam Kasten, San Mateo, CA (US); Daniel Lau, Oakland, CA (US); Sylvan Seth Canales, San Jose, CA (US); Daniel Shalom Eliahu, Alamo, CA (US); Miroslav Drahos, Lakewood, CO (US)

(73) Assignee: AURIS HEALTH, INC., Santa Clara, CA (US)

(21) Appl. No.: 19/006,134

(22) Filed: Dec. 30, 2024

Related U.S. Application Data
(60) Provisional application No. 63/571,603, filed on Mar. 29, 2024.

(51) Int. Cl. A61B 34/00; A61B 34/10; G06T 7/33; G06T 7/73; G06T 15/08; G06T 19/00; G06T 19/20; G16H 30/40; G16H 50/50

(52) U.S. Cl. CPC A61B 34/25; A61B 34/10; G06T 7/344; G06T 7/75; G06T 15/08; G06T 19/003; G06T 19/20; G16H 30/40; G16H 50/50; A61B 2034/105; A61B 2034/107; G06T 2200/24; G06T 2207/20092; G06T 2207/30004; G06T 2207/30064; G06T 2210/21; G06T 2210/41; G06T 2219/2004; G06T 2219/2016

(57) ABSTRACT

This disclosure provides methods, devices, and systems for planning and performing medical procedures. The present implementations more specifically relate to techniques for identifying an object (such as a medical instrument or a nodule) in an anatomy based on image data representing a three-dimensional (3D) model of the anatomy. In some aspects, a controller for a medical system may filter the image data based on known properties of the object (such as an expected image intensity distribution associated with the object) and generate a volumetric view of the anatomy based on the filtered image data so that the object is highlighted in the volumetric view. The controller further displays a graphical user interface (GUI) that includes the volumetric view of the anatomy and determines a pose of the object within the anatomy based on one or more user inputs received via the GUI.

### CROSS-REFERENCE TO RELATED APPLICATIONS

This application claims priority and benefit under 35 U.S.C. § 119 (e) to U.S. Provisional Patent Application No. 63/571,603, filed Mar. 29, 2024, which is incorporated herein by reference in its entirety.

### TECHNICAL FIELD

This disclosure relates generally to medical systems, and specifically to an instrument and target selection interface for registering an imaging system with a sensor system.

### DESCRIPTION OF RELATED ART

Many medical procedures include steps that can be performed pre-operation (also referred to as a “preoperative phase”), intra-operation (also referred to as an “intraoperative phase”), or post-operation (also referred to as a “postoperative phase”). For example, during a preoperative phase, an imaging system may be used to scan or otherwise capture images or video of a patient's anatomy. Example suitable imaging technologies include computed tomography (CT), X-ray, fluoroscopy, positron emission tomography (PET), PET-CT, CT angiography, cone beam CT (CBCT), three-dimensional rotational angiography (3DRA), single-photon emission CT (SPECT), magnetic resonance imaging (MRI), optical coherence tomography (OCT), and ultrasound, among other examples. The images may be used, during an intraoperative phase, to help guide or navigate a medical instrument to a target (also referred to as a “treatment site”) within the patient's anatomy. However, images acquired during a preoperative phase may not accurately reflect a spatial relationship between the medical instrument and the target during an intraoperative phase. For example, among various other factors, preoperative images are often acquired several days (or even weeks) before the intraoperative phase, such that changes in the patient's anatomy may cause deviations in the spatial positioning of the target. Thus, there is a need to provide more accurate information about the spatial relationship between the medical instrument and the target during the intraoperative phase.

### SUMMARY

This Summary is provided to introduce in a simplified form a selection of concepts that are further described below in the Detailed Description. This Summary is not intended to identify key features or essential features of the claimed subject matter, nor is it intended to limit the scope of the claimed subject matter.

One innovative aspect of the subject matter of this disclosure can be implemented in a method for identifying objects in an anatomy. The method includes steps of receiving image data representing a three-dimensional (3D) model of the anatomy; filtering the image data based at least in part on one or more known properties of an object within the anatomy; generating a volumetric view of the anatomy based on the filtered image data so that the object is highlighted in the volumetric view; displaying a graphical user interface (GUI) that includes the volumetric view of the anatomy; receiving one or more user inputs associated with the GUI; and determining a position of the object within the anatomy based on the one or more user inputs.

Another innovative aspect of the subject matter of this disclosure can be implemented in a controller for a medical system, including a processing system and a memory. The memory stores instructions that, when executed by the processing system, cause the controller to receive image data representing a 3D model of the anatomy; filter the image data based at least in part on one or more known properties of an object within the anatomy; generate a volumetric view of the anatomy based on the filtered image data so that the object is highlighted in the volumetric view; display a GUI that includes the volumetric view of the anatomy; receive one or more user inputs associated with the GUI; and determine a position of the object within the anatomy based on the one or more user inputs.

### BRIEF DESCRIPTION OF THE DRAWINGS

The present implementations are illustrated by way of example and are not intended to be limited by the figures of the accompanying drawings.

FIG. 1 shows an example medical system, according to some implementations.

FIG. 2 shows example components of the control system and the robotic system of FIG. 1, according to some implementations.

FIG. 3 shows a block diagram of an example localization system, according to some implementations.

FIG. 4 shows a block diagram of an example registration system, according to some implementations.

FIG. 5 shows a block diagram of an example system for identifying objects in an anatomy, according to some implementations.

FIG. 6 shows an example graphical user interface (GUI) usable for identifying a medical instrument in an anatomy, according to some implementations.

FIGS. 7A and 7B shows example primary and thumbnail displays depicting cross-sectional views of an anatomy.

FIG. 8 shows an example GUI usable for identifying a target in an anatomy, according to some implementations.

FIG. 9 shows an example cross-sectional view of an anatomy displayed in a GUI, according to some implementations.

FIG. 10A shows another example GUI usable for identifying objects in an anatomy, according to some implementations.

FIG. 10B shows another example GUI usable for identifying objects in an anatomy, according to some implementations.

FIG. 11 shows another example GUI usable for identifying objects in an anatomy, according to some implementations.

FIG. 12 shows another example GUI usable for identifying objects in an anatomy, according to some implementations.

FIG. 13 shows a block diagram of an example controller for a medical system, according to some implementations.

FIG. 14 shows an illustrative flowchart depicting an example operation for identifying objects in an anatomy, according to some implementations.

### DETAILED DESCRIPTION

In the following description, numerous specific details are set forth such as examples of specific components, circuits, and processes to provide a thorough understanding of the present disclosure. The term “coupled” as used herein means connected directly to or connected through one or more intervening components or circuits. The terms “electronic system” and “electronic device” may be used interchangeably to refer to any system capable of electronically processing information. Also, in the following description and for purposes of explanation, specific nomenclature is set forth to provide a thorough understanding of the aspects of the disclosure. However, it will be apparent to one skilled in the art that these specific details may not be required to practice the example implementations. In other instances, well-known circuits and devices are shown in block diagram form to avoid obscuring the present disclosure. Some portions of the detailed descriptions which follow are presented in terms of procedures, logic blocks, processing and other symbolic representations of operations on data bits within a computer memory.

These descriptions and representations are the means used by those skilled in the data processing arts to most effectively convey the substance of their work to others skilled in the art. In the present disclosure, a procedure, logic block, process, or the like, is conceived to be a self-consistent sequence of steps or instructions leading to a desired result. The steps are those requiring physical manipulations of physical quantities. Usually, although not necessarily, these quantities take the form of electrical or magnetic signals capable of being stored, transferred, combined, compared, and otherwise manipulated in a computer system. It should be borne in mind, however, that all of these and similar terms are to be associated with the appropriate physical quantities and are merely convenient labels applied to these quantities.

Unless specifically stated otherwise as apparent from the following discussions, it is appreciated that throughout the present application, discussions utilizing the terms such as “accessing,” “receiving,” “sending,” “using,” “selecting,” “determining,” “normalizing,” “multiplying,” “averaging,” “monitoring,” “comparing,” “applying,” “updating,” “measuring,” “deriving” or the like, refer to the actions and processes of a computer system, or similar electronic computing device, that manipulates and transforms data represented as physical (electronic) quantities within the computer system's registers and memories into other data similarly represented as physical quantities within the computer system memories or registers or other such information storage, transmission or display devices.

Certain standard anatomical terms of location may be used herein to refer to the anatomy of animals, and namely humans, with respect to the example implementations. Although certain spatially relative terms, such as “outer,” “inner,” “upper,” “lower,” “below,” “above,” “vertical,” “horizontal,” “top,” “bottom,” and similar terms, are used herein to describe a spatial relationship of one element, device, or anatomical structure to another device, element, or anatomical structure, it is understood that these terms are used herein for ease of description to describe the positional relationship between elements and structures, as illustrated in the drawings. It should be understood that spatially relative terms are intended to encompass different orientations of the elements or structures, in use or operation, in addition to the orientations depicted in the drawings. For example, an element or structure described as “above” another element or structure may represent a position that is below or beside such other element or structure with respect to alternate orientations of the subject patient, element, or structure, and vice-versa. As used herein, the term “patient” may generally refer to humans, anatomical models, simulators, cadavers, and other living or non-living objects.

In the figures, a single block may be described as performing a function or functions; however, in actual practice, the function or functions performed by that block may be performed in a single component or across multiple components, or may be performed using hardware, using software, or using a combination of hardware and software. To clearly illustrate this interchangeability of hardware and software, various illustrative components, blocks, modules, circuits, and steps have been described below generally in terms of their functionality. Whether such functionality is implemented as hardware or software depends upon the particular application and design constraints imposed on the overall system. Skilled artisans may implement the described functionality in varying ways for each particular application, but such implementation decisions should not be interpreted as causing a departure from the scope of the present disclosure. Also, the example systems or devices may include components other than those shown, including well-known components such as a processor, memory and the like.

The techniques described herein may be implemented in hardware, software, firmware, or any combination thereof, unless specifically described as being implemented in a specific manner. Any features described as modules or components may also be implemented together in an integrated logic device or separately as discrete but interoperable logic devices. If implemented in software, the techniques may be realized at least in part by a non-transitory processor-readable storage medium including instructions that, when executed, performs one or more of the methods described herein. The non-transitory processor-readable data storage medium may form part of a computer program product, which may include packaging materials.

The non-transitory processor-readable storage medium may comprise random access memory (RAM) such as synchronous dynamic random-access memory (SDRAM), read only memory (ROM), non-volatile random access memory (NVRAM), electrically erasable programmable read-only memory (EEPROM), FLASH memory, other known storage media, and the like. The techniques additionally, or alternatively, may be realized at least in part by a processor-readable communication medium that carries or communicates code in the form of instructions or data structures and that can be accessed, read, or executed by a computer or other processor.

The various illustrative logical blocks, modules, circuits and instructions described in connection with the implementations disclosed herein may be executed by one or more processors (or a processing system). The term “processor,” as used herein may refer to any general-purpose processor, special-purpose processor, conventional processor, controller, microcontroller, or state machine capable of executing scripts or instructions of one or more software programs stored in memory.

As described above, many medical procedures include a preoperative phase that precedes an intraoperative phase. During the preoperative phase, for some medical procedures, an imaging system may be used to scan or otherwise capture images or video of at least a portion of a patient's anatomy. For example, a computed tomography (CT) scanner may be used to acquire tomographic images (also referred to as “tomograms” or “CT scans”) of a patient's lungs during the preoperative phase for a bronchoscopy. A tomogram is a cross-section or slice of a three-dimensional (3D) volume. For example, multiple tomograms can be stacked or combined to recreate the 3D volume (such as a 3D model of the patient's lungs). Thus, tomograms can be used to detect a precise location or position (in 3D space) of a nodule or target in the patient's lungs. During the intraoperative phase, for some medical procedures, a medical system may use the preoperative images to generate a graphical interface for navigating a medical instrument within the patient's anatomy. For example, during a bronchoscopy, the medical system may detect a pose of an endoscope (such as a position and orientation of the scope in 3D space) based on sensor data received via an electromagnetic (EM) sensor disposed on the tip of the scope and map the pose of the endoscope to a 3D model of the patient's lungs depicted by the tomograms.

Accordingly, the graphical interface may depict a spatial relationship between the medical instrument and the target within the anatomy based on the sensor data and the image data. However, images acquired during a preoperative phase may not accurately reflect the spatial relationship between the medical instrument and the target during an intraoperative phase. For example, changes in the patient's anatomy or the medical environment can cause the spatial relationship between the endoscope and target to deviate from what is depicted by the graphical interface at any given time, which can lead to inaccurate navigation. Example factors include EM distortion, poor registration (or mapping) between the sensor space and the image space associated with the preoperative scans (also referred to as the “preoperative image space”), outdated preoperative scans, and anatomical deformations, among other examples. Aspects of the present disclosure recognize that some modern imaging technologies (such as cone beam CT) can be used to scan a patient's anatomy during an intraoperative phase. In some aspects, a medical system may capture updated images of the patient's anatomy during the intraoperative phase and use the updated image data to improve the representation of the spatial relationship between the medical instrument and the target. The updated image data and sensor data are often associated with different coordinate spaces. Thus, in some implementations, the medical system may “register” the updated image space with the sensor space to facilitate real-time navigation.

As used herein, the term “registration” refers to a mapping or transformation between different coordinate spaces. For example, a medical system (or registration system associated therewith) may register an imaging system used for capturing images of a patient's anatomy (such as a cone beam CT scanner) with a sensor system used for tracking a pose of a medical instrument within the anatomy (such as an EM field generator) by determining a mapping or spatial transformation that maps any point or vector in the image space to a respective point or vector in the sensor space (such as a transformation matrix). The terms “mapping,” “transformation,” “spatial transformation,” and “registration matrix,” may be used interchangeably herein. The terms “respective” and “corresponding” also may be used interchangeably herein,

Although certain aspects of the present disclosure are described in detail herein in the context of bronchoscopy, it should be understood that the systems and techniques of the present disclosure may be applicable to any medical procedure. Example medical procedures may include minimally invasive procedures (such as laparoscopy), non-invasive procedures (such as endoscopy), therapeutic procedures, diagnostic procedures, percutaneous procedures, and non-percutaneous procedures, among other examples. Example endoscopic procedures include bronchoscopy, ureteroscopy, gastroscopy, nephroscopy, and nephrolithotomy, among other examples. The terms “scope,” “endoscope,” “catheter,” and “instrument” may be used interchangeably herein.

Aspects of the present disclosure may be used to perform robotic-assisted medical procedures, such as endoscopic access, percutaneous access, or treatment for a target anatomical site. For example, robotic tools may engage or control one or more medical instruments (such as an endoscope) to access a target site within a patient's anatomy or perform a treatment at the target site. In some implementations, the robotic tools may be guided or controlled by a physician. In some other implementations, the robotic tools may operate in an autonomous or semi-autonomous manner. Although systems and techniques are described herein in the context of robotic-assisted medical procedures, the systems and techniques may be applicable to other types of medical procedures (such as procedures that do not rely on robotic tools or only utilize robotic tools in a very limited capacity). For example, the systems and techniques described herein may be applicable to medical procedures that rely on manually operated medical instruments (such as an endoscope that is exclusively controlled and operated by a physician). The systems and techniques described herein also may be applicable beyond the context of medical procedures (such as in simulated environments or laboratory settings, such as with models or simulators, among other examples).

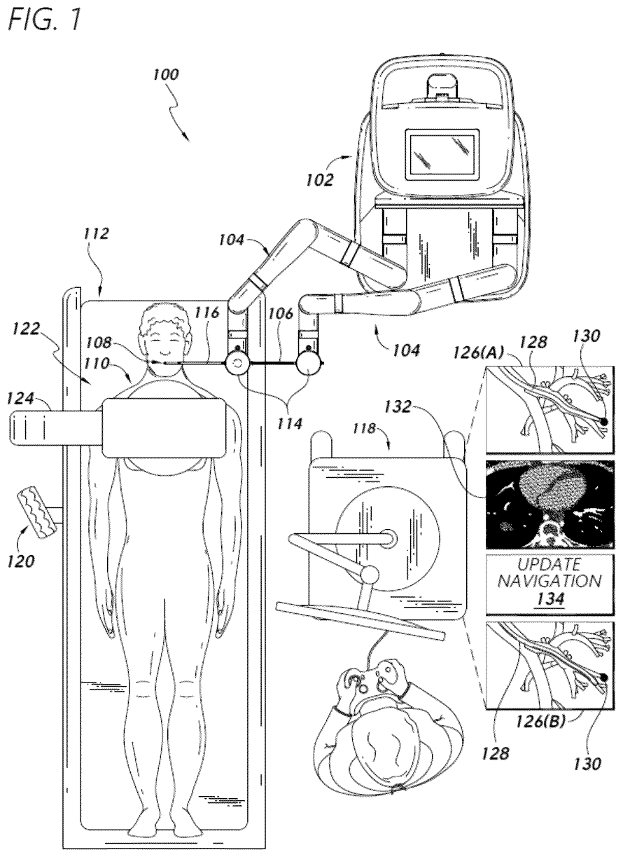

FIG. 1 shows an example medical system 100 (also referred to as a “surgical medical system” or a “robotic medical system”), according to some implementations. As shown in FIG. 1, the medical system 100 may be arranged for diagnostic or therapeutic bronchoscopy. The medical system 100 can include and utilize a robotic system 102 which can be implemented, for example, as a robotic cart. Although the medical system 100 is shown as including various cart-based systems or devices, the concepts disclosed herein can be implemented in any type of robotic system or arrangement, such as robotic systems employing rail-based components, table-based robotic end-effectors, or manipulators, among other examples. The robotic system 102 may include one or more robotic arms 104 (also referred to as “robotic positioners”) configured to position or otherwise manipulate a medical instrument 106 (such as a steerable endoscope or another elongate instrument). For example, the medical instrument 106 can be advanced through a natural orifice access point (such as the mouth 108 of a patient 110 positioned on a table 112) to deliver diagnostic or therapeutic treatment. Although described in the context of a bronchoscopy procedure, the medical system 100 also may be used to perform other types of medical procedures. Example suitable procedures include gastro-intestinal (GI) procedures, renal procedures, urological procedures, and nephrological procedures, among other examples.

With the robotic system 102 properly positioned, the medical instrument 106 can be inserted into the patient 110 robotically, manually, or a combination thereof. For example, the one or more robotic arms 104, or instrument drivers 114 coupled thereto, can control the medical instrument 106. In some implementations, the medical instrument 106 may be advanced within a sheath 116. For example, the sheath 116 may be coupled to, or controlled by, a robotic arm 104. In some implementations, the medical instrument 106 and the sheath 116 may each be coupled to a respective instrument driver from a set of instrument drivers 114. The instrument drivers 114 can be repositionable in space by manipulating the one or more robotic arms 104 into different angles or positions.

In the example of FIG. 1, the medical instrument 106 can be directed down the patient's trachea and lungs after insertion or advanced to a target destination or operative site. In some implementations, to enhance navigation through the patient's lung network or reach the desired target, the medical instrument 106 may be manipulated to telescopically extend from the outer sheath 116 to obtain enhanced articulation or greater bend radius. The use of separate instrument drivers 114 can allow the medical instrument 106 and sheath 116 to be driven independently of each other.

In some implementations, the medical instrument 106 may include an elongate member or shaft configured to be inserted or retracted, articulated, or otherwise moved within the anatomy. Further, in some implementations, the medical instrument 106 may include one or more imaging devices (such as cameras) positioned on a distal end of the elongate shaft or deployed through a working channel of the elongate shaft. The imaging devices can be configured to generate or capture image (or video) data or send the image data to another device or component. In some implementations, the medical instrument 106 may include an instrument base or one or more handles positioned at a proximal end of the medical instrument 106. The instrument base can be coupled to a manipulator (such as an end of a robotic arm 104). The instrument base can include one or more drive inputs coupled to one or more drive outputs of the manipulator, wherein the drive inputs or drive outputs act as an interface.

In some implementations, the medical instrument 106 may include a working channel configured to receive one or more other instruments or elements therein or provide other functionality. The working channel can extend axially, such as along the length of the medical instrument 106. Furthermore, the medical instrument 106 can include or be associated with one or more elongate movement members (such as pulls wires) that can extend from a proximal end through the elongate shaft to the distal end of the elongate shaft. The elongate movement members can be manipulated, such as by manipulators on the one or more robotic arms 104, to control actuation of the elongate movement members.

In some implementations, the medical instrument 106 may include one or more sensors, such as electromagnetic (EM) sensors, shape sensors (such as shape sensing fiber), accelerometers, gyroscopes, satellite-based positioning sensors (such as global positioning system (GPS) sensors), or radio-frequency (RF) transceivers, among other examples. The sensors can be configured to generate or produce sensor data or provide the sensor data to another device or component. The sensors can be disposed at a distal end of the elongate shaft or along a length of the elongate shaft. In some implementations, the medical instrument 106 may be configured to receive an elongate member or device through a working channel, wherein the elongate member includes one or more sensors along a length of the elongate member. One or more sensors on the medical instrument 106 may provide sensor data to control circuitry of the medical system 100, which is then used to determine a position, orientation, or shape of the medical instrument 106.

The medical system 100 can also include a control system 118 (also referred to as a “control tower” or “mobile tower”). The control system 118 can be communicatively coupled (such as via wired or wireless connections) to the robotic system 102 to control various aspects of the robotic system 102 (such as electronics, optics, sensors, or power) or one or more subsystems associated with the robotic system 102, such as a fluid management system (not shown). Placing such functionality in the control system 118 can allow for a smaller form factor of the robotic system 102 that may be more easily adjusted or re-positioned by an operator or user. Additionally, the division of functionality between the robotic system 102 and the control system 118 can reduce operating room clutter and facilitate efficient clinical workflow.

The medical system 100 can include an electromagnetic (EM) field generator 120, which is configured to broadcast or emit an EM field that can be detected by various EM sensors, such as a sensor disposed on the medical instrument 106. The EM field can induce small electric currents in coils of the EM sensors, which can be analyzed to determine a position, angle, or orientation of the EM sensors relative to the EM field generator 120. Although EM fields and EM sensors are described in many examples herein, position sensing systems or sensors can include various other types of position sensing systems or sensors, such as optical position sensing systems or sensors, image-based position sensing systems or sensors, among other examples.

The medical system 100 can further include an imaging system 122 (also referred to as an “imaging device”) configured to generate, provide, or send image data (also referred to as “images”) to another device or system. For example, the imaging system 122 can generate image data depicting an anatomy of the patient 110 and provide the image data to the control system 118, the robotic system 102, or another device. The imaging system 122 may include an emitter or energy source (such as an X-ray source) or a detector (such as an X-ray detector) mounted on a C-shaped arm support 124, which allows for flexibility in positioning around the patient 110 to capture images from various angles without moving the patient 110. Use of the imaging system 122 can provide visualization of internal structures or anatomy, which can be used for a variety of purposes, including navigation of the medical instrument 106 (such as by providing images of internal anatomy to a user) and localization of the medical instrument 106 (based on an analysis of image data), among other examples. In some aspects, the imaging system 122 may enhance the efficacy or safety of a medical procedure, such as a bronchoscopy, by providing clear, continuous visual feedback to the operating surgeon or team.

In some implementations, the imaging system 122 may be a mobile device configured to move around an environment. For example, the imaging system 122 can be positioned next to the patient 110 (as shown in FIG. 1) during a particular phase of a procedure and removed when the imaging system 122 is no longer needed. In some other implementations, the imaging system 122 may be part of the table 112 or other equipment in an operating environment. The imaging system 122 can be implemented as a Computed Tomography (CT) machine or system, X-ray machine or system, fluoroscopy machine or system, Positron Emission Tomography (PET) machine or system, PET-CT machine or system, CT angiography machine or system, Cone-Beam CT (CBCT) machine or system, three-dimensional rotational angiography (3DRA) machine or system, single-photon emission computed tomography (SPECT) machine or system, Magnetic Resonance Imaging (MRI) machine or system, Optical Coherence Tomography (OCT) machine or system, or ultrasound machine or system, among other examples. In some implementations, the medical system 100 may include different types of imaging systems that can be used or positioned over the patient 110 during different phases or portions of a procedure depending on the needs at that time.

In some implementations, the imaging system 122 may be configured to process multiple images (also referred to as “image data”) to generate a three-dimensional (3D) view or model. For example, the imaging device 122 can be implemented as a CT machine configured to capture or generate a series of images (also referred to as “tomograms) or image data representing two-dimensional (2D) cross-sections or slices of a 3D volume from different angles around the patient 110, and then use one or more algorithms to reconstruct these images or image data into a 3D model. The 3D model can be provided to the control system 118, robotic system 102, or another device, such as for processing or display.

In some implementations, image data from the imaging system 122 may be used to localize various elements, such as the medical instrument 106, a target within the anatomy, or specific anatomical features, among other examples. As used herein, the terms “localize,” “localization,” or “localizing” refer to any processes for determining or estimating a position (or location) and/or orientation (or heading), collectively referred to as the “pose,” of the instrument or the target (or any other element) within a given space or environment. For example, the control system 118 can be configured to provide navigation information during a procedure to assist a user navigating the medical instrument 106 within the anatomy to reach a target (such as a desired treatment site or location). In some implementations, a target can include a nodule, such as in the context of certain bronchoscopy procedures. To illustrate, the control system 118 can display a navigation view or graphical data 126 that includes an instrument indicator 128 representing the medical instrument 106, a target indicator 130 representing the target, and an anatomical map. The navigation data 126(A) (such as initial navigation data) can be determined based on sensor data from a sensor of the medical instrument 106 (such as EM sensor data associated with the EM field generator 120), a map of the anatomy, or a location of the target. In some implementations, the map or location of the target may be determined based on preoperative data, such as data obtained during a preoperative procedure to find a target location or map the anatomy.

In some implementations, the navigation data 126(A) may be dynamically updated based on image data 132 from the imaging system 122. For example, the control system 118 can receive the image data 132 and analyze the image data 132 to determine a current or actual spatial relationship between the medical instrument 106 and the target. In some implementations, the control system 118 may display the image data 132 to a user, receive user input indicating a position of the medical instrument 106 or a position of the target in the image data 132, and analyze the image data 132 based on the user input to determine the current spatial relationship. If the control system 118 determines that the navigation data 126(A) incorrectly depicts the location of the medical instrument 106 (such as where the spatial relationship associated with the image data 132 is different than the spatial relationship associated with the navigation data 126(A)), the control system 118 may update the navigation data 126(A) at 134 and provide updated navigation data 126(B) that reflects the current or near real-time position of the medical instrument 106 relative to the target or the map.

The various components of the medical system 100 can be communicatively coupled to each other over a network, which can include a wireless or wired network. Example networks include one or more personal area networks (PANs), local area networks (LANs), wide area networks (WANs), Internet area networks (IANs), cellular networks, the Internet, personal area networks (PANs), body area network (BANs), etc. In some examples, various communication interfaces can include wireless technology, such as Bluetooth, Wi-Fi, near-field communication (NFC), or the like. Furthermore, in some examples, the various components of the medical system 100 can be connected for data communication, fluid exchange, power exchange, and so on, via one or more support cables, tubes, connections, or the like.

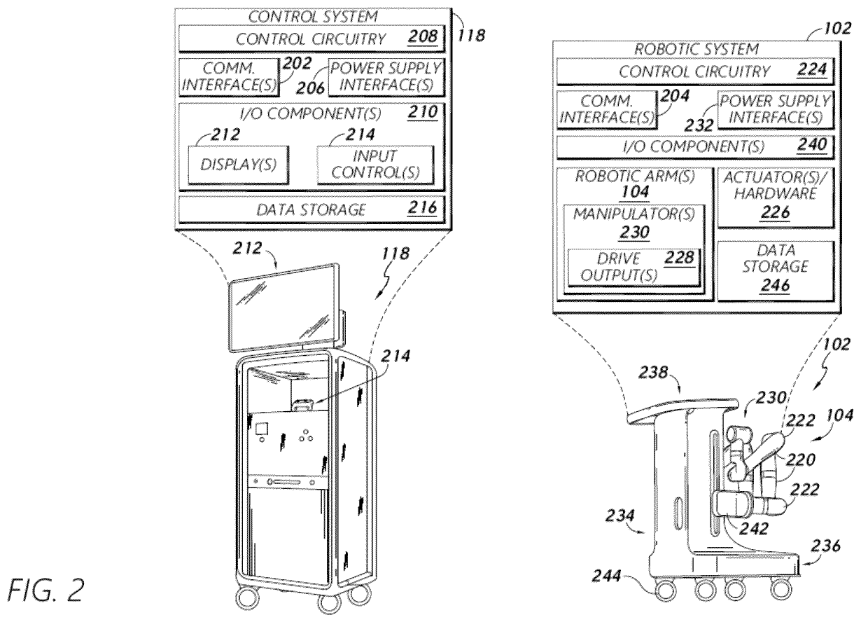

FIG. 2 shows example components of the control system 118 and the robotic system 102 of FIG. 1, according to some implementations. In the examples of FIG. 2, the control system 118 and the robotic system 102 are implemented as a tower and a robotic cart, respectively. However, the control system 118 and robotic system 102 can be implemented in other manners. The control system 118 can be coupled to the robotic system 102 and operate in cooperation therewith to perform a medical procedure. For example, the control system 118 can include communication interface(s) 202 for communicating with communication interface(s) 204 of the robotic system 102 via a wireless or wired connection (such as to control the robotic system 102). In some implementations, the control system 118 may communicate with the robotic system 102 to receive position or sensor data therefrom relating to the position of sensors associated with an instrument or member controlled by the robotic system 102. For example, the control system 118 may communicate with the EM field generator 120 to control generation of an EM field in an area around a patient. The control system 118 can further include one or more power supply interface(s) 206.

The control system 118 can include control circuitry 208 configured to cause one or more components of the medical system 100 to actuate or otherwise control any of the various system components, such as carriages, mounts, arms or positioners, medical instruments, imaging devices, position sensing devices, or sensors, among other examples. Further, the control circuitry 208 can be configured to perform other functions, such as cause display of information, process data, receive input, communicate with other components or devices, or any other function or operation described herein.

The control system 118 can further include one or more input or out (I/O) components 210 configured to assist a physician or others in performing a medical procedure. For example, the one or more I/O components 210 can be configured to receive input or provide output to enable a user to control or navigate the medical instrument 106, the robotic system 102, or other instruments or devices associated with the medical system 100. The control system 118 can include one or more displays 212 to provide, display or otherwise present various information regarding a procedure. For example, the one or more displays 212 can be used to present navigation information including a virtual anatomical model of anatomy with a virtual representation of a medical instrument, image data, or other information. The one or more I/O components 210 can include one or more user input control(s) 214, which can include any type of user input (or output) devices or device interfaces, such as one or more buttons, keys, joysticks, handheld controllers (such as video-game-type controllers), computer mice, trackpads, trackballs, control pads, sensors (such as motion sensors or cameras) that capture hand gestures and finger gestures, touchscreens, toggle (such as button) inputs, or interfaces or connectors therefore. In some implementations, such inputs can be used to generate commands for controlling one or more medical instruments, robotic arms, or other components.

The control system 118 can also include data storage 216 configured to store executable instruments (such as computer-readable instructions) that can be executed by the control circuitry 208 to cause the control circuitry 208 to perform various operations or functionality described herein. In some implementations, the data storage 216 also may store telemetry or runtime data (such as sensor data or image data) generated by the medical system 100 or otherwise captured or acquired during a medical procedure. In some implementations, two or more components of the control system 118 can be electrically or communicatively coupled to each other.

The robotic system 102 can include the one or more robotic arms 104 configured to engage with or control, for example, the medical instrument 106 or other elements or components to perform one or more aspects of a procedure. As shown in FIG. 2, each robotic arm 104 can include multiple segments 220 coupled to joints 222, which can provide multiple degrees of movement or freedom. The robotic system 102 can be configured to receive control signals from the control system 118 to perform certain operations, such as to position one or more of the robotic arms 104 in a particular manner or manipulate an instrument, among other examples. In response, the robotic system 102 can control, using control circuitry 224 thereof, actuators 226 or other components of the robotic system 102 to perform the operations. For example, the control circuitry 224 can control insertion or retraction, articulation, or roll of a shaft of the medical instrument 106 or other instrument by actuating one or more drive outputs 228 of a manipulator 230 (or end-effector) coupled to a base of a robotically-controllable instrument. The drive outputs 228 can be coupled to a drive input on an associated instrument, such as an instrument base of an instrument that is coupled to the associated robotic arm 104. The robotic system 102 also may include one or more power supply interfaces 232.

The robotic system 102 can include a support column 234, a base 236, or a console 238. The console 238 can provide one or more I/O components 240, such as a user interface for receiving user input or a display screen (or a dual-purpose device, such as a touchscreen) to provide the physician or user with preoperative or intraoperative data. The support column 234 can include an arm support 242 (also referred to as a “carriage”) for supporting the deployment of the one or more robotic arms 104. The arm support 242 can be configured to vertically translate along the support column 234. Vertical translation of the arm support 242 allows the robotic system 102 to adjust the reach of the robotic arms 104 to meet a variety of table heights, patient sizes, or physician preferences. The base 236 can include wheel-shaped casters 244 (also referred to as “wheels”) that allow the robotic system 102 to move around the operating room. After reaching the appropriate position, the casters 244 can be immobilized using wheel locks to hold the robotic system 102 in place during the procedure.

The joints 222 of each robotic arm 104 can each be independently-controllable or provide an independent degree of freedom available for instrument navigation. In some implementations, each robotic arm 104 may include seven joints that provide seven degrees of freedom, including “redundant” degrees of freedom. Redundant degrees of freedom can allow robotic arms 104 to be controlled to position their respective manipulators 230 at a specific position, orientation, or trajectory in space using different linkage positions and joint angles. This allows for the robotic system 102 to position or direct a medical instrument from a desired point in space while allowing the physician to move the joints 222 into a clinically advantageous position away from the patient to create greater access, while avoiding collisions.

The one or more manipulators 230 (or end-effectors) can be coupled to an instrument base or handle, which can be attached using a sterile adapter component. The combination of the manipulator 230 and instrument base, as well as any intervening mechanics or couplings (such as the sterile adapter), can be collectively referred to as the manipulator or a manipulator assembly. Manipulators or manipulator assemblies can provide power or control interfaces. Example interfaces may include connectors to transfer pneumatic pressure, electrical power, electrical signals, or optical signals from the robotic arm 104 to an instrument base. Manipulators or manipulator assemblies can be configured to manipulate medical instruments (such as surgical tools) using techniques including, for example, direct drives, harmonic drives, geared drives, belts or pulleys, or magnetic drives, among other examples.

The robotic system 102 can also include data storage 246 configured to store executable instruments (such as computer-readable instructions) that can be executed by the control circuitry 224 to cause the control circuitry 224 to perform various operations or functionality described herein. In some implementations, the data storage 216 also may store telemetry or runtime data (such as sensor data or image data) generated by the medical system 100 or otherwise captured or acquired during a medical procedure. In some implementations, two or more of the components of the robotic system 102 can be electrically or communicatively coupled to each other.

Data storage (including the data storage 216, data storage 246, or other data storage or memory) can include any suitable or desirable type of computer-readable media. For example, computer-readable media can include one or more volatile data storage devices, non-volatile data storage devices, removable data storage devices, or nonremovable data storage devices implemented using any technology, layout, or data structure(s) or protocol, including any suitable or desirable computer-readable instructions, data structures, program modules, or other types of data.

Computer-readable media that can include, but is not limited to, phase change memory, static random-access memory (SRAM), dynamic random-access memory (DRAM), other types of random access memory (RAM), read-only memory (ROM), electrically erasable programmable read-only memory (EEPROM), flash memory or other memory technology, compact disk read-only memory (CD-ROM), digital versatile disks (DVD) or other optical storage, magnetic cassettes, magnetic tape, magnetic disk storage or other magnetic storage devices, or any other non-transitory medium that can be used to store information for access by a computing device. As used in certain contexts herein, computer-readable media may not generally include communication media, such as modulated data signals and carrier waves. As such, computer-readable media should generally be understood to refer to non-transitory media.

Functionality described herein can be implemented by the control circuitry 208 of the control system 118 or the control circuitry 224 of the robotic system 102, such as by the control circuitry 208 or 224 executing instructions to cause the control circuitry 208 or 224 to perform the functionality. Control circuitry (including the control circuitry 208, control circuitry 224, or other control circuitry) can include circuitry embodied in a robotic system, control system or tower, instrument, or any other component or device. Control circuitry can include any collection of processors, processing circuitry, processing modules or units, chips, dies (such as semiconductor dies including one or more active or passive devices or connectivity circuitry), microprocessors, micro-controllers, digital signal processors, microcomputers, central processing units, field-programmable gate arrays, programmable logic devices, state machines (such as hardware state machines), logic circuitry, analog circuitry, digital circuitry, or any device that manipulates signals (analog or digital) based on hard coding of the circuitry or operational instructions.

Control circuitry referenced herein can further include one or more circuit substrates (such as printed circuit boards), conductive traces and vias, or mounting pads, connectors, or components. Control circuitry can further include one or more storage devices, which may be embodied in a single device, a plurality of devices, or embedded circuitry of a device. Such data storage can comprise read-only memory, random access memory, volatile memory, non-volatile memory, static memory, dynamic memory, flash memory, cache memory, data storage registers, or any device that stores digital information. In examples in which control circuitry includes a hardware or software state machine, analog circuitry, digital circuitry, or logic circuitry, data storage device(s) or register(s) storing any associated operational instructions can be embedded within, or external to, the circuitry comprising the state machine, analog circuitry, digital circuitry, or logic circuitry.

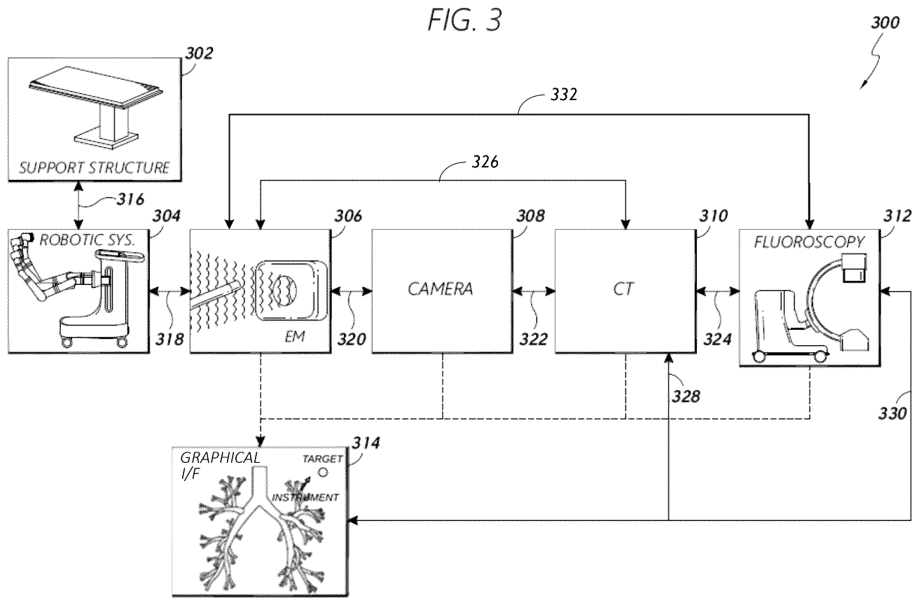

FIG. 3 shows a block diagram of an example localization system 300, according to some implementations. The localization system 300 includes various positioning or imaging systems or modalities 302-312 (also referred to as “subsystems”), which can be implemented to facilitate anatomical mapping, navigation, positioning, or visualization for procedures in accordance with one or more examples. For example, the various systems 302-312 can be configured to provide data for generating an anatomical map, determining a location of an instrument, determining a location of a target, or performing other techniques.

Each of the systems 302-312 can be associated with a respective coordinate space (also referred to as a “position coordinate frame”) or can provide data or information relating to instrument or anatomy locations, wherein registering the various coordinate spaces to one another can allow for integration of the various systems to provide mapping, navigation, or instrument visualization. For example, registering a first modality to a second modality can allow for determined positions in the first modality to be tracked or superimposed on or in a reference frame associated with the second modality, thereby providing layers of positional information that can be combined to provide a robust localization system.

In some aspects, the system 300 may be configured to perform one or more localization or localizing techniques. In some implementations, the anatomical space in which a medical instrument can be localized (such as where a pose or shape of the instrument is determined or estimated) may be a 2D or 3D portion of a patient's tracheobronchial airways, vasculature, urinary tract, gastrointestinal tract, or any organ or space accessed via lumens. Various modalities can be implemented to provide images, representations, or models of the anatomical space. For example, an imaging modality can be implemented, which can include, for example, X-ray, fluoroscopy, CT, PET, PET-CT, CT angiography, CBCT, 3DRA, SPECT, MRI, OCT, or ultrasound, among other examples. In some implementations, the imaging modality may be used to capture or acquire images of a patient's anatomy during a preoperative phase of a medical procedure. In some other implementations, the imaging modality may be used to capture or acquire images of a patient's anatomy during an intraoperative phase of the medical procedure.

The systems 302-312 can provide information for generating a graphical user interface 314 (also referred to as a “graphical interface (I/F)”) that includes navigation information for navigating an instrument to a target within an anatomy (such as the navigation data 126(A) or 126(B) of FIG. 1). For example, the navigation information may include an anatomical map, an estimated position, orientation, and/or shape of the instrument. The navigation information also may include a shape, boundary, eccentricity, texture, and/or position of the target. In some implementations, the graphical user interface 314 or other localization information may be displayed to a user, such as a physician, during a medical procedure to assist the user in performing the procedure. For example, a visualization of a tracked instrument can be superimposed on an anatomical map depicted by the graphical user interface 314 based on position or sensor data associated with the tracked medical instrument.

As shown in FIG. 3, the system 300 can include a support structure 302 (such as a surgical bed or other patient positioning or support platform). For example, the support structure 302 includes a planar surface that contacts and supports the patient. In some implementations, the position of the support structure 302 may be known based on data maintained relating to the position of the support structure 302 within the surgical or procedure environment. In some other implementations, the position of the support structure 302 may be sensed or otherwise determined using one or more markers or an appropriate imaging or positioning modality.

The system 300 can further include a robotic system 304 (such as a robotic cart or other device or system including one or more robotic end effectors). In some implementations, the robotic system 304 may be one example of the robotic system 102 of FIGS. 1 and 2. Data relating to the position or state of robotic arms, actuators, or other components of the robotic system 304 can be known or derived from robotic command data or other robotic data relative to a coordinate frame of the robotic system 304. In some examples, reference frame registration 316 occurs between the support structure 302 and the robotic system 304, which can be a relatively coarse registration, in some implementations, based on robotic system or cart-set-up procedure (which can have any suitable or desirable scheme).

The system 300 can further include an electromagnetic (EM) sensor system 306, which can include an EM field generator (such as the EM field generator 120 of FIG. 1) and one or more EM sensors. An EM sensor can be associated with a portion of an instrument that is tracked or controlled, such as a distal end (or tip) of the instrument or along a length of the instrument or other elongate member (such as a working channel) disposed in a lumen of the instrument. In some implementations, the EM field generator can be mechanically coupled to the support structure 302 or the robotic system 304 such that registration or association 318 between such systems can be known or determined. In some implementations, the registration 318 between the EM sensor system 306 and the robotic system 304 can be determined through forward kinematics or field generator mount transform information. For example, the field generator can be mounted to the support structure 302 such that the position of the field generator can be known relative to the robotic system positioning frame based on a known relationship between the position of the support structure 302 and the robotic system 304. The EM sensor system 306 can provide instrument pose or path information based on sensor readings associated with the instrument.

The system 300 can further include an optical camera system 308 including one or more cameras or other imaging devices configured to generate images of patient anatomy within a visual field thereof (such as real-time image data) during a surgical procedure. In some implementations, registration 320 between the optical camera system 308 and the EM sensor system 306 can be achieved through identification of features having EM sensor data associated therewith, such as a medical instrument tip, in images generated by the optical camera system 308. The registration 320 can further be based at least in part on hand-eye interaction of the physician when viewing real-time camera images while the EM-sensor-equipped endoscope is navigating in the patient anatomy.

The system 300 can further include a computed tomography (CT) imaging system 310 configured to generate CT images of the patient anatomy, which can be performed preoperatively or intraoperatively. In some implementations, image processing can be implemented for registration 322 of the CT image data with the camera image data generated by the optical camera system 308. For example, common features identified in both camera image data and CT image data can be identified to relate the CT image frame to the camera image frame in space. In some examples, the CT imaging system 310 can be used to generate preoperative imaging data for producing the graphical user interface 314 or for path navigation planning.

In some aspects, the CT imaging system 310 may be registered 326 to the EM sensor system 306 through various techniques, such as tool registration or a transformation function, among other examples. In some implementations, a mechanical structure of the CT imaging system 310 can have a known physical transform or relationship with respect to a mounting position of the EM field generator of the EM sensor system 306. Such known relationship can be used to register the CT image space to the EM sensor space. The connection 328 represents a mapping or relationship between the CT imaging system 310 and an anatomical map depicted by a graphical user interface 314.

The system 300 can further include a fluoroscopy imaging system 312 configured to generate tomographic images (such as real-time X-ray images) of the surgical site. In some implementations, the fluoroscopy imaging system 312 may be one example of the imaging system 122 of FIG. 1. For example, the fluoroscopy imaging system 312 may include a CBCT scanner coupled to a C-arm. In some implementations, the fluoroscopy imaging system 312 may be used with a contrast agent introduced into the anatomy to generate image data representing patient anatomy or instrumentation. In some implementations, the fluoroscopy imaging system 312 may be registered 324 to the CT imaging system 310 using any image processing technique suitable for such registration.

In some aspects, the fluoroscopy imaging system 312 may be registered 332 to the EM sensor system 306 through various techniques, such as tool registration or a transformation function, among other examples. In some implementations, a mechanical structure of the fluoroscopy imaging system 312 (such as the C-arm instrumentation) can have a known physical transform or relationship with respect to a mounting position of the EM field generator of the EM sensor system 306. Such known relationship can be used to register the fluoroscopy image space to the EM sensor space. The connection 330 represents a mapping or relationship between the fluoroscopy imaging system 312 and an anatomical map depicted by the graphical user interface 314.

In the example of FIG. 3, the CT imaging system 310 and fluoroscopy imaging system 312 are illustrated as separated systems. However, in some other implementations, a single imaging system may perform the functions of both the CT imaging system 310 and fluoroscopy imaging system 312.

The position, shape, or orientation of an instrument, such as an endoscope, can be determined using any one or more of the systems 302-312, which can facilitate generation of graphical interface data representing the estimated position or shape of the instrument relative to an anatomical map depicted by the graphical user interface 314. The graphical user interface 314 can be displayed on a display device, such as via the control system 118 or robotic system 102, or another device. In some implementations, the graphical user interface 314 also may indicate a position of a target within the anatomy that has been designated for treatment.

Although the systems 302-312 have been described in a particular order, the operations or functions associated therewith can be performed in different orders. In some implementations, the systems 302-312 can be used in different ways. In some other implementations, registration can occur between different systems and modalities.

In some aspects, one or more of the systems 302-312 may be used to generate the graphical user interface 314 preoperatively or determine a location of one or more targets within an anatomical map depicted by the graphical user interface 314 during a preoperative phase of a medical procedure. However, a graphical user interface 314 generated using a preoperative CT scan may not accurately reflect the spatial relationship between a medical instrument and a target during an intraoperative phase. For example, changes in the patient's anatomy or the medical environment can cause the spatial relationship between the instrument and the target to deviate from what is depicted in the graphical user interface 314. Example factors that may cause such deviations include EM distortion, poor registration (or mapping) between the sensor data and the image data, outdated preoperative scans, and anatomical deformations, among other examples. Thus, in some other aspects, one or more of the systems 302-312 may be used to determine a location of a medical instrument or position of a target relative to an anatomical map depicted by the graphical user interface 314 during an intraoperative phase of the medical procedure.

As described with reference to FIG. 3, image data and sensor data are often associated with different coordinate spaces. For example, the image data may describe data points (such as coordinates or vectors) in relation to a coordinate space defined by an imaging system (such as the fluoroscopy imaging system 312) whereas the sensor data may describe data points (such as coordinates or vectors) in relation to a coordinate space defined by a sensing system (such as the EM sensor system 306). To combine the sensor data and the image data on a single frame of reference (such as the graphical user interface 314), the system 300 may register the coordinate space associated with the image data (also referred to as the “image space”) with the coordinate space associated with the sensor data (also referred to as the “sensor space”). As used herein, the term “registration” refers to a mapping between different coordinate spaces. For example, the system 300 (or a registration system associated therewith) may register 332 the fluoroscopy imaging system 312 with the EM sensor system 306 by determining a mapping or spatial transformation that maps any data point in the image space to a respective data point in the sensor space. The system 300 may further use the registration 332 to dynamically update the graphical user interface 314 to reflect the current or actual spatial relationship between the instrument and the target during the intraoperative phase, as depicted by the image data captured via the fluoroscopy imaging system 312 (such as to facilitate real-time navigation).

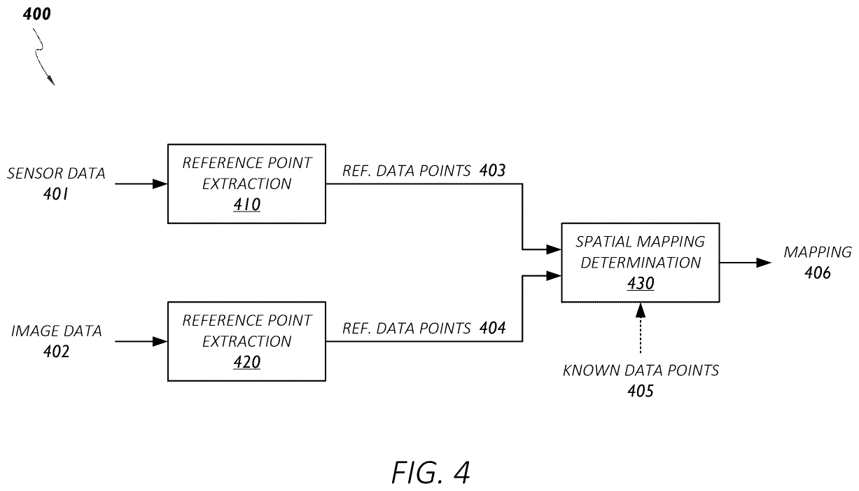

FIG. 4 shows a block diagram of an example registration system 400, according to some implementations. In some implementations, the registration system 400 may be one example of any of the control circuitry 208 or 224 of FIG. 2. The registration system 400 is configured to register an imaging system with a sensor system, or vice-versa, during an intraoperative phase of a medical procedure to facilitate real-time navigation of a medical instrument within an anatomy. In some implementations, the sensor system and the imaging system may be examples of the EM sensor system 306 and the fluoroscopy imaging system 312, respectively, of FIG. 3.

In some aspects, the registration system 400 may determine a mapping 406 between a coordinate space associated with the sensor system and a coordinate space associated with the imaging system based, at least in part, on sensor data 401 and image data 402 received via the sensor system and the imaging system, respectively. For example, the mapping 406 may be used to transform any data point (such as a coordinate or a vector) in the image space to a respective data point in the sensor space. In some implementations, the sensor data 401 may include position or heading information about one or more EM sensors disposed in an EM field (such as described with reference to FIGS. 1-3). In some implementations, the image data 402 may include one or more tomograms captured by a CBCT scanner (such as described with reference to FIGS. 1-3). Aspects of the present disclosure recognize that some imaging technologies (such as CT or X-rays) may interfere with some sensor technologies (such as EM). In some aspects, the registration system 400 may determine the mapping 406 based on sensor data 401 that is captured or acquired while the imaging system cannot interfere with the underlying sensor technology (such as when a CBCT system is located outside an EM field).

The registration system 400 includes a first reference point extraction component 410, a second reference point extraction component 420, and a spatial mapping determination component 430. The first reference point extraction component 410 is configured to determine one or more reference data points 403 based on the sensor data 401 and the second reference point extraction component 420 is configured to determine one or more reference data points 404 based on the image data 402. As used herein, the term “reference data point” may refer to any object, feature, point, or vector that can be identified or found in both the sensor space and the image space. In some implementations, the reference data points 403 and 404 may include a position of a medical instrument (such as a position of the tip of an endoscope). In some other implementations, the reference data points 403 and 404 may include a heading of the medical instrument (such as an orientation of the tip of the endoscope).

For example, the first reference point extraction component 410 may determine the position and heading of an endoscope (in the sensor space) based on sensor data 401 associated with an EM sensor disposed on or coupled to the tip of the endoscope. The second reference point extraction component 420 may determine the position and heading of the same scope tip (in the image space) through analysis of the image data 402 using one or more image processing techniques. Example suitable image processing techniques include segmentation, machine learning, and statistical analysis, among other examples. As used herein, the term “segmentation” refers to various techniques for partitioning a digital image into groups of voxels (or “image segments”) based on related characteristics or identifying features. In some implementations, the second reference point extraction component 420 may segment the image data 402 so that the position and heading of the scope tip can be detected or estimated from the corresponding tomograms.

The spatial mapping determination component 430 is configured to determine the mapping 406 based, at least in part, on the reference data points 403 in the sensor space and the reference data points 404 in the image space. In some implementations, the mapping 406 may be a transformation matrix. Aspects of the present disclosure recognize that at least 3 unique reference data points (which may include any combination of coordinates or vectors) are needed to define a unique transformation matrix in 3D space. For example, given 3 unique reference data points, the spatial mapping determination component 430 can determine a transformation matrix that transforms the 3 reference data points in the image space to the 3 reference data points in the sensor space (or vice-versa) using various known techniques or algorithms. Example suitable algorithms for determining the spatial transformation matrix include singular value decomposition (SVD) and the iterative closest point (ICP) algorithm, among other examples.

In some implementations, each of the reference point extraction components 410 and 420 may be configured to extract at least 3 unique reference data points 403 and 404 from the sensor data 401 and the image data 402, respectively. In such implementations, the spatial mapping determination component 430 may determine the mapping 406 using only the reference data points 403 and 404. However, in some other implementations, at least one of the reference point extraction components 410 or 420 may provide fewer than 3 unique reference data points 403 or 404, respectively. For example, the reference data points 403 may include only the position or heading of the instrument in the sensor space and the reference data points 404 may include only the position or heading of the instrument in the image space. In such implementations, the spatial mapping determination component 430 may rely on one or more known data points 405 in the image space and the sensor space to determine the mapping 406.

As described with reference to FIG. 4, some implementations of the registration system 400 may use the pose of the instrument (including position and orientation) in the image space and the pose of the instrument in the sensor space to determine the mapping 406 between the image space and the sensor space. In some implementations, the reference point extraction component 420 may determine the pose of the instrument in the image space by segmenting the image data 402. However, existing segmentation techniques cannot ensure the accuracy of their estimates. Thus, in some implementations, the reference point extraction component 420 may rely on the professional judgment of a user or operator of the medical system (such as a physician or a technician) to determine the instrument pose in the image space. For example, the reference point extraction component 420 may display a graphical user interface (GUI) with various tools and/or features for selecting or otherwise specifying a pose of an instrument (or various other objects) in the image space.

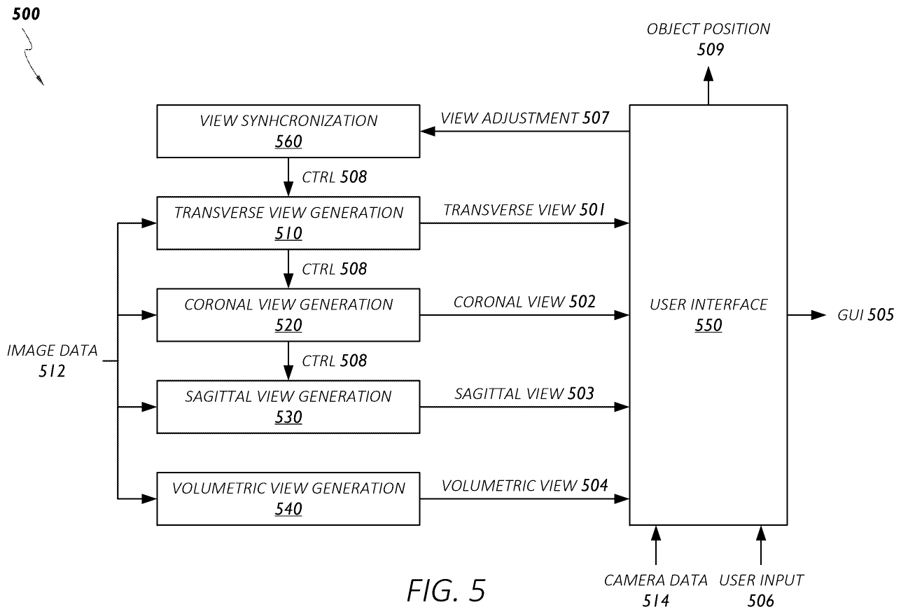

FIG. 5 shows a block diagram of an example system 500 for identifying objects in an anatomy, according to some implementations. More specifically, the system 500 is configured to determine a position 509 of an object (such as an instrument or a target) within an anatomy based at least in part on image data 512 received from an imaging system. In some implementations, the system 500 may be one example of the reference point extraction component 420 of FIG. 4. With reference to FIG. 4, the image data 512 may be one example of the image data 402 and the object position 509 may be one example of any of the reference data points 404. For example, the image data 512 may include tomograms representing a three-dimensional (3D) model of an anatomy. In some aspects, the system 500 may provide a graphical user interface (GUI) 505 to guide or facilitate user input 506 for selecting or otherwise specifying the position of the object 509. For example, the GUI 505 may be displayed on a display device (such as any of the one or more displays 212 of FIG. 2).

The system 500 includes a transverse view generation component 510, a coronal view generation component 520, a sagittal view generation component 530, a volumetric view generation component 540, a user interface component 550, and a view synchronization component 560. Each of the components 510-530 is configured to generate a respective cross-sectional view of the anatomy based on the image data 512. More specifically, the transverse view generation component 510 is configured to generate a cross-sectional view 501 in a transverse plane of the anatomy (also referred to as the “transverse view”), the coronal view generation component 520 is configured to generate a cross-sectional view 502 in a coronal plane of the anatomy (also referred to as the “coronal view”), and the sagittal view generation component 530 is configured to generate a cross-sectional view 503 in a sagittal plane of the anatomy (also referred to as the “sagittal view”).

The volumetric view generation component 540 is configured to generate a volumetric (or 3D) view 504 of the anatomy based at least in part on the image data 512. In some aspects, the volumetric view generation component 540 may filter the image data 512 based at least in part on one or more known properties of the desired object so that the object is highlighted in the volumetric view 504. In some implementations, the known properties of the object may include an expected image intensity distribution. For example, aspects of the present disclosure recognize that some objects within an anatomy, such as medical instruments or nodules, may be denser than the surrounding tissue. As a result, such objects may appear brighter than the surrounding anatomy in x-ray images (such as those captured by CT or CBCT imaging systems). In some implementations, the volumetric view generation component 540 may filter the image data 512 by hiding any voxels having brightness or intensity values below a threshold intensity associated with the density of the desired object.

The user interface component 550 is configured to arrange the cross-sectional views 501-503 and the volumetric view 504 for display or presentation in the GUI 505. In some implementations, the user interface component 550 also may display a “camera view” in the GUI 505 based on real-time image data 514 received from a camera disposed on an instrument within the anatomy (also referred to as “camera data”). For example, the camera view may provide a live view of the anatomy based on the current instrument pose. In some implementations, the user interface component 550 may display one of the cross-sectional views 501-503 in a “primary” display region of the GUI 505 and may display each of the remaining cross-sectional views in a respective “thumbnail” display region of the GUI 505, where the primary display region is presented more prominently in the GUI 505 than any of the thumbnail display regions. For example, the primary display region may occupy a larger portion of the GUI 505, or may be outlined in a different color, than the thumbnail display regions. In some implementations, the user interface component 550 may add a virtual plane to the volumetric view 504 that intersects the anatomy at the cross-section displayed in the primary display region of the GUI 505.

The user interface component 550 is also configured to receive user inputs 506 via one or more input devices (not shown for simplicity). Example suitable input devices may include touchscreens, touchpads, buttons, switches, mice, keyboards, keypads, joysticks, and scroll wheels, among other examples. In some implementations, the user interface component 550 may dynamically switch the cross-sectional view presented in the primary display region of the GUI 505 with a cross-sectional view presented in one of the thumbnail display regions responsive to user input 506. For example, in response to a user tapping, clicking, or otherwise selecting one of the thumbnail display regions of the GUI 505, the user interface component 550 may move the cross-sectional view displayed in the selected thumbnail display region to the primary display region of the GUI 505 and move the cross-sectional view that was previously displayed in the primary display region to the selected thumbnail display region of the GUI 505.

In some aspects, the user interface 550 may display an interactive reticle in the GUI 505 that can be used to select the desired object. For example, a user may drag the reticle (via one or more user inputs 506) in the GUI 505 so that it is centered on the desired object. In some implementations, the GUI 505 may display instructions for using the reticle to select the desired object. The reticle may be displayed in all three cross-sectional views 501-503 to identify a particular point or coordinate in the 3D image space. In some implementations, the user interface component 550 may add a positioning feature to the volumetric view 504 (such as a crosshair disposed on the virtual plane) that indicates the position of the reticle in 3D space. In some aspects, a user may change the position of the reticle only in the primary display region of the GUI 505. In other words, the reticle may be displayed as a static feature in the thumbnail display regions of the GUI 505. In some implementations, the cross-sectional views displayed in each of the thumbnail display regions of the GUI 505 may be centered around the reticle. For example, as the user drags the reticle around in the primary display region of the GUI 505, the user interface component 550 may adjust the cross-sectional views displayed in the thumbnail display regions to maintain alignment with the position of the reticle.

Aspects of the present disclosure recognize that, due to the orthogonality of the cross-sectional views 501-503, changing the position of the reticle in one of the planes may require updating the tomographic images or cross-sections of the anatomy (also referred to as “slices”) in the orthogonal planes. In some implementations, the user interface component 550 may provide view adjustment information 507 to the view synchronization component 560 indicating the position of the reticle in relation to the cross-sectional view displayed in the primary display region of the GUI 505. The view synchronization component 560 may provide control (CTRL) signals 508 to one or more of the cross-sectional view generation components 510-530 to provide updated cross-sectional views of the anatomy based on the reticle position. For example, if the transverse view 501 is currently displayed in the primary display region of the GUI 505, the view synchronization component 560 may cause the coronal view generation component 520 and/or the sagittal view generation component 530 to produce updated cross-sectional views 502 and/or 503, respectively, based on the movement of the reticle.

In some aspects, the GUI 505 may include one or more features (such as sliders) for adjusting the cross-sectional views 501-503 based on one or more user inputs 506. Example suitable features may include, among other examples, a brightness slider for changing a brightness (or window level) of the displayed content and a contrast slider for changing a contrast (or window width) of the displayed content. In some implementations, adjusting the brightness slider or the contrast slider may change the brightness or contrast for one of the cross-sectional views 501-503 (such as the cross-sectional view currently displayed in the primary display region of the GUI 505). In some other implementations, adjusting the brightness slider or the contrast slider may change the brightness or contrast of all of the cross-sectional views 501-503, concurrently.

In some aspects, the GUI 505 may include one or more features (such as sliders) that can be used for uniquely adjusting the content displayed in the primary display region of the GUI 505. Example suitable features may include, among other examples, a zoom slider for changing a magnification of the cross-sectional view displayed in the primary display region of the GUI 505 and a slice slider for changing a depth of the cross-sectional plane or slice of the anatomy depicted in the primary display region. In some implementations, the magnification may be centered on the position of the reticle. For example, a user may interact with the zoom slider to zoom in (or out) on the position of the reticle in the primary display region of the GUI 505. In some implementations, the user interface component 550 may update the cross-sectional views in the thumbnail display regions of the GUI 505 to track the degree of magnification in the primary display region. In such implementations, user inputs 506 associated with the magnification slider may change the magnification in all of the cross-sectional views 501-503, concurrently.

In some implementations, the user interface component 550 may provide view adjustment information 507 to the view synchronization component 560 indicating a degree and direction of movement of the slice slider. The view synchronization component 560 may provide control signals 508 to one of the cross-sectional view generation components 510-530 to provide updated cross-sectional views of the anatomy based on the movement of the slice slider. For example, if the transverse view 501 is currently displayed in the primary display region of the GUI 505, the view synchronization component 560 may cause the transverse view generation component 520 to produce a new transverse cross-sectional view 501 representing a new tomographic image or cross-section of the anatomy associated with the degree and direction of movement of the slice slider. Because the position of the reticle is mapped to the slice of the anatomy displayed in the primary display region of the GUI 505, changing the cross-sectional view in the primary display region also changes the position of the reticle in relation to the anatomy. Thus, as a user adjusts the slice slider for the primary display region of the GUI 505, the user interface 550 may shift or otherwise move around the cross-sectional views displayed in the thumbnail display of the regions to maintain alignment with the position of the reticle.

In some aspects, the user interface component 550 may display the volumetric view 504 as an interactive feature in the GUI 505. For example, a user may drag the volumetric view 504 (via one or more user inputs 506) in the GUI 505 to rotate the volumetric view 504 around one or more axes. In some implementations, the GUI 505 may include an automated alignment feature for aligning the reticle with a desired point in the volumetric view 504. More specifically, the automated alignment feature may align the position of the reticle and the cross-sectional views 501-503, displayed in each of the display regions of the GUI 505, to any user-selected position in the volumetric view 504. For example, a user may tap or click on an estimated position of the object in the volumetric view 504 (via one or more user inputs 506) to cause the user interface 550 to align the reticle and the cross-sectional views 501-503 displayed in the GUI 505 with the estimated position of the object. As such, the automated alignment feature may significantly streamline the workflow for the user and reduce the amount of time required to locate or identify the desired object in the 3D image space.

The GUI 505 may further include a feature that can be used to finalize a selection of the object. For example, once the user is satisfied with their selection of the object, the user may tap or click on a button (via one or more user inputs 506) that locks in the current reticle position as the position of the object in the image space.

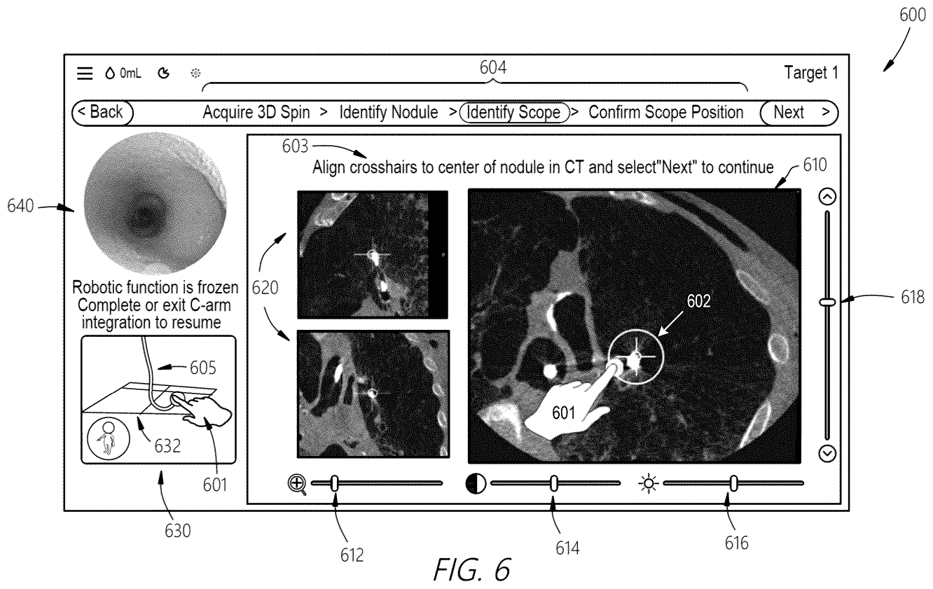

FIG. 6 shows an example GUI 600 usable for identifying a medical instrument 605 in an anatomy, according to some implementations. In the example of FIG. 6, the medical instrument 605 is depicted as an endoscope. However, in actual implementations, the GUI 600 may be used to identify various other types of medical instruments. In some implementations, the GUI 600 may be one example of the GUI 505 of FIG. 5. With reference for example to FIG. 5, the GUI 600 may be generated by the system 500 based at least in part on tomographic image data (such as the image data 512).

The GUI 600 includes a primary display region 610, thumbnail display regions 620, a volumetric display region 630, and a camera display region 640. With reference for example to FIG. 5, the primary display region 610 may display one of the cross-sectional views 501-503, the thumbnail display regions 620 may display the remaining cross-sectional views that are not displayed in the primary display region 610, the volumetric display region 630 may display the volumetric view 504, and the camera display region 640 may display real-time images (or video) of the anatomy based on the camera data 514.

In some aspects, a user may tap, click, or otherwise provide user input indicating a selection of one the thumbnail display regions 620 to switch the content of the selected thumbnail display region 620 with the content of the primary display region 610. For example, a user may tap on the upper thumbnail display region 620 to move the cross-sectional view currently displayed in the upper thumbnail display region 620 to the primary display region 610 and to move the cross-sectional view currently display in the primary display region 610 to the upper thumbnail display region 620.

The primary display region 610 includes an interactive reticle 602 that can be used to select a position of the instrument 605 in response to user input 601. As shown in FIG. 6, the GUI 600 also includes instructions 603 to the user for selecting the position of the instrument 605. For example, a user may drag the reticle 602 to any desired position within the primary display region 610. In the example of FIG. 6, user inputs 601 are depicted as a finger touching the GUI 600 (such as a touchscreen input device). However, in actual implementations, the user inputs 601 may be provided via any suitable input device (including touchpads, buttons, switches, mice, keyboards, keypads, joysticks, and scroll wheels, among other examples).

In some implementations, the position of the reticle 602 may be displayed as a static display feature in the thumbnail display regions 620. In other words, a user may not adjust or otherwise interact with the reticle displayed in any of the thumbnail display regions 620. However, due to the orthogonality of the cross-sectional planes, the GUI 600 may update the cross-sectional views displayed in the thumbnail display regions 620 in response to movements of the reticle 602 in the primary display region 610, for example, to maintain alignment with the reticle position (such as described with reference to FIG. 5).

The volumetric display region 630 provides an interactive 3D model of the anatomy that responds to user input 601. For example, a user may drag the 3D model to rotate the model around one or more axes. A figurine is displayed in the lower left corner of the volumetric display region 630 to indicate the relative (isometric) view of the anatomy. As shown in FIG. 6, the volumetric display region 630 includes a virtual plane 632 that intersects the anatomy at the cross-section depicted by the cross-sectional view in the primary display region 610. The virtual plane 632 further includes a pair of orthogonal lines (also referred to as a “crosshair”) that intersect at the position of the reticle 602. Thus, as the user moves the reticle 602 around, the GUI 600 also may adjust the position of the crosshairs on the virtual plane 632.

In the example of FIG. 6, the 3D model of the anatomy is filtered so that only the endoscope can be seen in the volumetric display region 630. For example, the filter may hide any voxels of image data having an intensity below a threshold value. Because an endoscope is generally denser than any anatomical features, a threshold can be selected so that only the endoscope appears (in isolation) in the 3D model. This allows a user to identify the position of the scope tip more quickly. In some implementations, a user may tap, click, or otherwise select a point on the volumetric display region 630 (such as an estimated position of the distal end of the instrument 605) to cause the GUI 600 to align the position of the reticle 602 (including adjusting the cross-sectional views displayed in the primary display region 610 and the thumbnail display regions 620) with the selected point (such as described with reference to FIG. 5).

The GUI 600 also includes a number of sliders 612-618 that can be used to manually adjust the content of the primary display region 610 and the thumbnail display regions 620 (such as by tapping or dragging any of the sliders 612-618). In the example of FIG. 6, the GUI 600 includes a contrast slider 614 that can be used to adjust a contrast (or window width) of the cross-sectional views and a brightness slider 616 that can be used to adjust a brightness (or window level) of one or more of the cross-sectional views. The GUI 600 also includes a zoom slider 612 that can be used to magnify the cross-sectional view in the primary display region 610, for example, by zooming in (or out) on the position of the reticle 602. In some implementations, the GUI 600 may adjust the magnification of the cross-sectional views in the thumbnail display regions 620 responsive to changes in the magnification of the cross-sectional view depicted in the primary display region 610 (such as described with reference to FIG. 5).

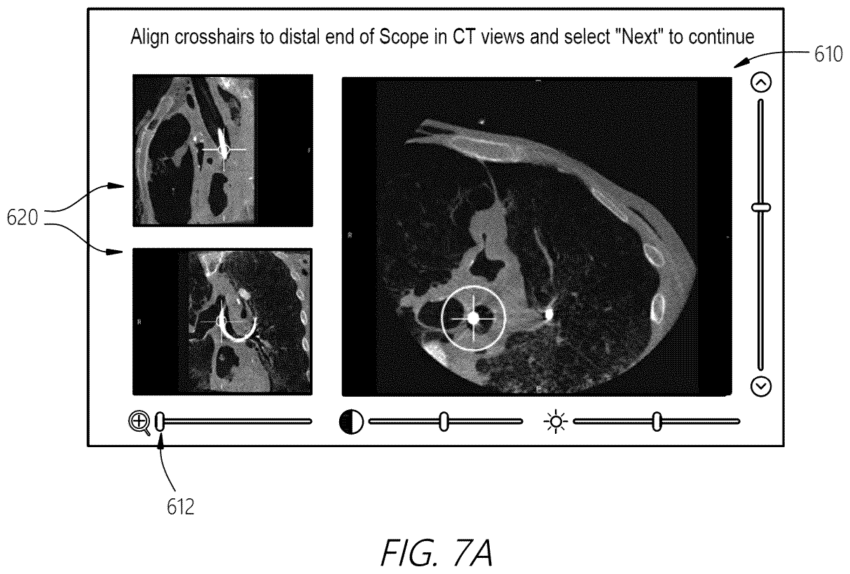

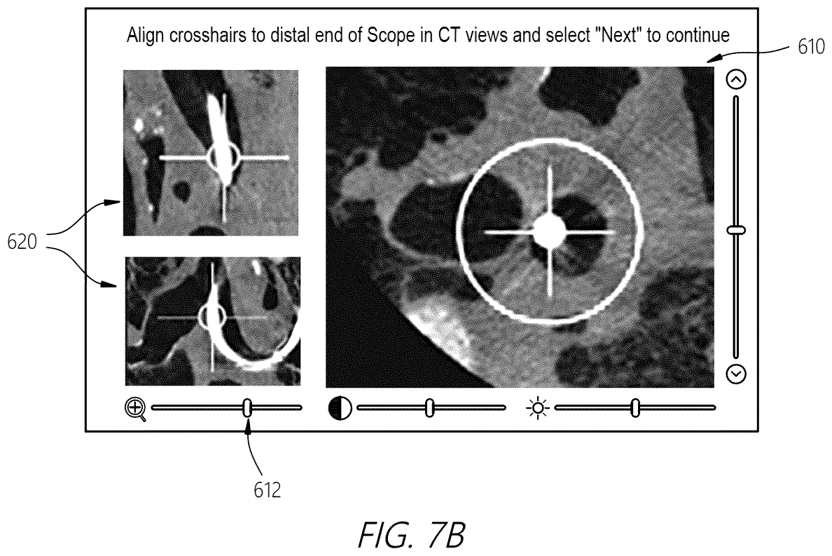

For example, FIGS. 7A and 7B show primary and thumbnail displays regions 610 and 620 depicting example cross-sectional views of an anatomy in response to user interaction with the zoom slider 612. More specifically, FIG. 7A shows the primary display region 610 and the thumbnail display regions 620 before the user makes any adjustments to the zoom slider 612 and FIG. 7B shows the primary display region 610 and the thumbnail display regions 620 after the user adjusts the zoom slider 612 to zoom in on the position of the reticle. As a result of the zoom operation, the reticle is more centrally positioned in the primary display region 610 shown in FIG. 7B compared to the position of the reticle relative to the primary display region 610 shown in FIG. 7A. As shown in FIGS. 7A and 7B, the content displayed in the thumbnail display regions 620 also changes in response to the movement of the zoom slider 612.

The GUI 600 also includes slice slider 618 that can be used to change the cross-section or slice of the anatomy displayed in the primary display region 610. For example, a user may drag the slider to make coarse adjustments to the depth (and direction) of the cross-section or tap on the up or down arrows to make fine adjustments to the depth of the cross-section in a particular direction. Because the thumbnail display regions 620 track the position of the reticle 602 in the primary display region 610, the GUI 600 may shift or move around the cross-sectional views displayed in one or more of the thumbnail display regions 620 in response to user interactions with the slice slider 618, for example, to maintain alignment with the reticle position (such as described with reference to FIG. 5).

In some aspects, the system 500 may analyze the image data 512 around the placement of the reticle 602 to estimate the pose of the instrument 605. For example, the user interface 550 may implement one or more image processing techniques (such as segmentation, machine learning, or statistical analysis) to determine a position and orientation of the instrument 605 in the 3D image space. In some implementations, the system 500 may provide a visual indicator in the GUI 600 indicating whether an instrument pose has been successfully estimated based on the current position of the reticle 602. In the example of FIG. 6, the visual indicator is depicted as a “Next” icon in the upper right corner of the GUI 600. For example, the “Next” icon may be highlighted or selectable (via user input) only after the system 500 has successfully estimated a pose of the instrument 605 based on the current position of the reticle 602.

Tapping or clicking on the “Next” icon locks in the current position of the reticle as the scope tip position for registration. In some implementations, clicking on the “Next” icon may cause the GUI 600 to proceed with a subsequent step of a workflow (such as to allow the user to confirm or adjust the pose of the instrument). In some other implementations, a user may click or tap on the “Back” icon in the upper left corner of the GUI 600 to revert to a previous workflow step (such as to determine or specify a position of the target). In some aspects, the GUI 600 may provide various other visual indications about whether an instrument pose has been successfully estimated in addition to, or in lieu of, the “Next” icon. For example, in some implementations, the GUI 600 may change a color of the reticle 602 to indicate that the system 500 has successfully estimated the instrument pose (such as by coloring the reticle 602 green) or was unable to estimate the instrument pose (such as by coloring the reticle 602 red).

Example workflow steps 604 associated with registering an image space to a sensor space are displayed at the top of the GUI 600, between the “Back” icon and the “Next” icon. In the example of FIG. 6, the workflow steps 604 may include “Acquire 3D Spin” (to capture or acquire intraoperative image data), “Identify Nodule” (to determine or specify a position of the target), “Identify Scope” (which is highlighted as the current workflow step), and “Confirm Scope Position” (to confirm or adjust the position and orientation of the instrument). In some implementations, the order of the workflow steps 604 may differ from the order shown in FIG. 6. In some other implementations, the workflow steps associated with a given registration procedure may differ (or include fewer or more steps) than the workflow steps 604 depicted in FIG. 6.

Various aspects of the GUI 600 may be customized to user preferences. Example suitable customization options may include, among other examples, changing the number or locations of the views relative to the GUI 600, changing the relative dimensions of one or more views, displaying one or more oblique views (which may not be orthogonal to the transverse, coronal, or sagittal planes), adjusting rendering parameters (such as color, opacity, or intensity of various features) of the volumetric view (such as via a transfer function editor), or changing a color used to highlight the primary display region of the GUI 600. Any specific text, fonts, shapes, buttons, icons, or other graphical features shown in the GUI 600 are merely for purposes of illustration and may be configured differently (or user-configurable) in actual implementations. For example, the “Next” icon may be replaced with “Set,” “Select,” or “Lock” in some other implementations.

In some implementations, the GUI 600 may include additional highlighting of the instrument 605 (such as a colored outline or shading, among other examples) in the primary display region 610 and/or the thumbnail display regions 620 to help distinguish the instrument 605 from the surrounding anatomy. In some other implementations, the GUI 600 may display additional text or instructions on or near each interactive control feature (such as any of the sliders 612-618, the reticle 602, and/or the volumetric view 630), when the GUI 600 is first presented to a user, to indicate the intended usage or effect of the feature. For example, such instructions may disappear in response to a user interacting with one or more of the control features. Still further, in some implementations, the GUI 600 may provide additional tools for characterizing a relationship (such as distances, angles, and trajectories, among other examples) between the instrument 605 and a target.

In some aspects, the GUI 600 may adjust one or more of the control features based on knowledge of the task to be completed by the user. In some implementations, the GUI 600 may programmatically adjust the brightness slider 612 and/or the contrast slider 614 based on the current image data (such as the image data 512 of FIG. 5) to optimize the cross-sectional views for instrument (or target) identification. In some other implementations, the GUI 600 may programmatically adjust a transfer function used for rendering the current image data (such as the image data 512 of FIG. 5) based on user-specified anatomy or objects of interest.

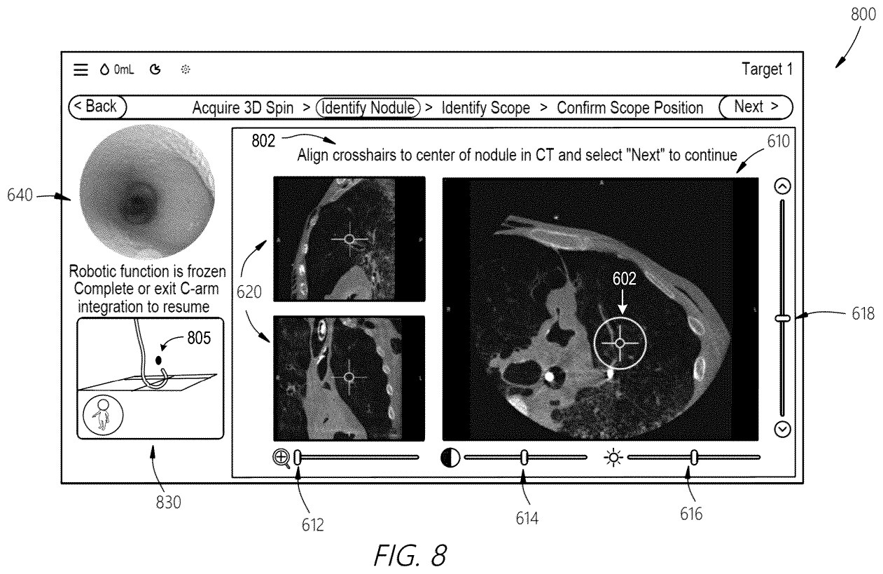

FIG. 8 shows an example GUI 800 usable for identifying a target 805 in an anatomy, according to some implementations. In the example of FIG. 8, the target 805 is depicted as a nodule. However, in actual implementations, the GUI 800 may be used to identify various other types of targets. In some implementations, the GUI 800 may be one example of the GUI 505 of FIG. 5. With reference for example to FIG. 5, the GUI 800 may be generated by the system 500 based at least in part on tomographic image data (such as the image data 512).

As shown in FIG. 8, GUI 800 includes many of the same features and functions as the GUI 600 of FIG. 6. For example, the GUI 800 includes the primary display region 610, the thumbnail display regions 620, the camera display region 640, the reticle 602, and the set of sliders 612-618 having the same features and functionality as described with reference to FIG. 6. However, unlike the GUI 600, the GUI 800 includes instructions 802 to a user for selecting the position of the target 805.

The GUI 800 also includes a volumetric display region 830 that differs from the volumetric display region 630 of FIG. 6. Aspects of the present disclosure recognize that nodules are generally denser than any surrounding soft tissue but may not be as dense as some other anatomical features (such as bone). Thus, in the example of FIG. 8, the 3D model of the anatomy is filtered so that the target 805 can be clearly seen in the volumetric display region 630 but is not displayed in isolation. For example, the volumetric display region 830 also shows the endoscope as well as some other anatomical features not shown in the volumetric display region 630 of FIG. 6. Similar to the volumetric display region 630 of FIG. 6, a user may tap or click on the target 805 shown in the volumetric display region 830 to cause the GUI 800 to align the position of the reticle 602 (including adjusting the cross-sectional views displayed in the primary display region 610 and the thumbnail display regions 620) with an estimated position of the target 805 (such as described with reference to FIG. 5).

When a user is satisfied with the alignment of the reticle 602 in each of the cross-sectional views, the user may tap or click on the “Next” icon in the upper right corner of the GUI 800. This locks in the current position of the reticle 602 as the nodule position for registration. In some implementations, clicking on the “Next” icon may cause the GUI 800 to proceed with a subsequent step of a workflow (such as determining a position of an endoscope).

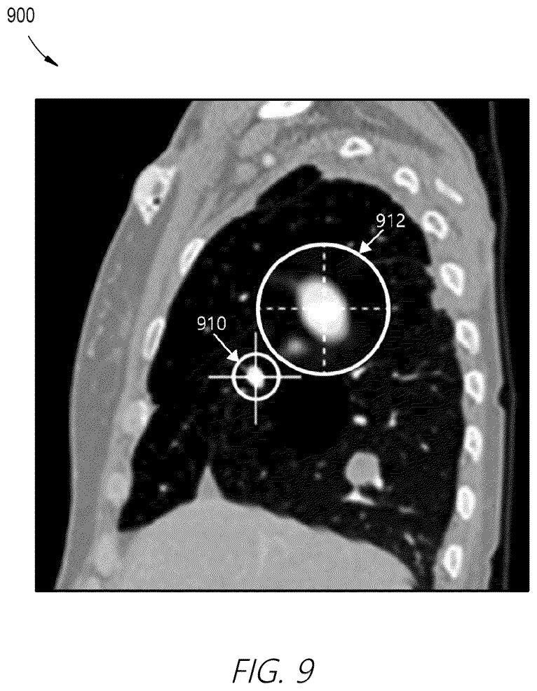

FIG. 9 shows an example cross-sectional view 900 of an anatomy displayed in a GUI, according to some implementations. In some implementations, the GUI may be one example of the GUI 600 of FIG. 6. With reference to FIG. 6, the cross-sectional view 900 may be one example of any of the cross-sectional views displayed in the primary display region 610 or the thumbnail display regions 620.

The cross-sectional view shows a reticle 910 and a magnifier 912 next to the reticle 910. In some implementations, the reticle 910 may be one example of the reticle 602 of FIG. 6. In the example of FIG. 9, the magnifier 912 is configured to provide local magnification of the content inside or encircled by the reticle 910. For example, the magnifier 912 may allow the user to quickly determine whether the reticle 910 is positioned over a desired object (such as an instrument or a target). In some implementations, the magnifier 912 may be provided as an additional feature of the GUI 600 to supplement the magnification slider 612. In some other implementations, the magnifier 912 may replace the magnification slider 612 of the GUI 600.

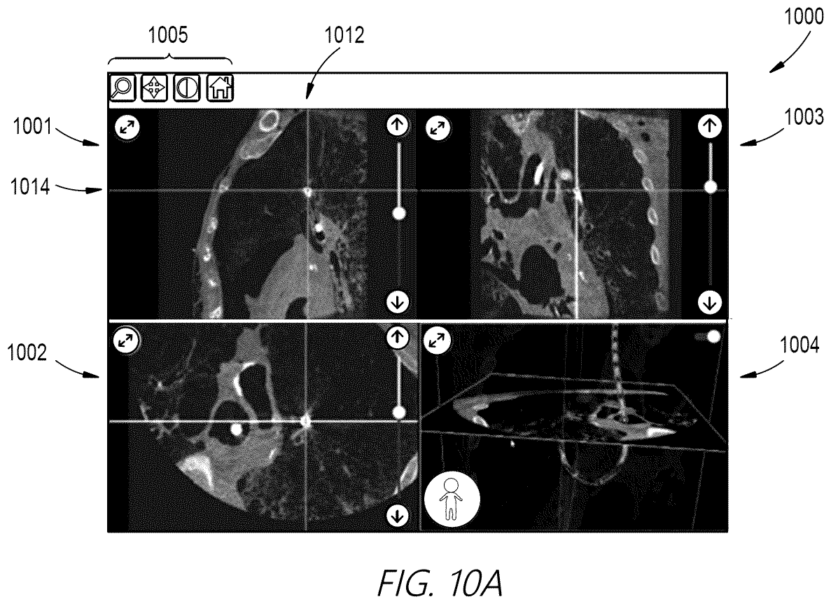

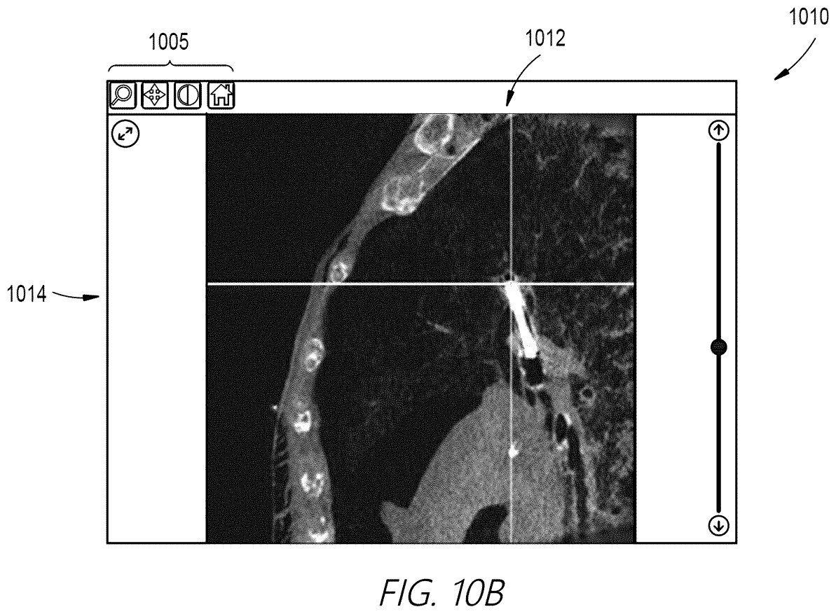

FIG. 10A shows another example GUI 1000 usable for identifying objects in an anatomy, according to some implementations. In some implementations, the GUI 1000 may be one example of the GUI 505 of FIG. 5. With reference for example to FIG. 5, the GUI 1000 may be generated by the system 500 based at least in part on tomographic image data (such as the image data 512).

The GUI 1000 includes 4 display regions 1001-1104 and a toolbar 1005. With reference for example to FIG. 5, each of the first three display regions 1001-1003 may display one of the cross-sectional views 501-503 and the fourth display region 1004 may display the volumetric view 504. Unlike the GUI 600 of FIG. 6, the GUI 1000 also does not include a primary display region (or thumbnail display regions). In other words, the GUI 1000 does not emphasize any particular display region over the others. Rather, a user may expand any particular one of the display regions 1001-1004 by tapping or clicking the arrow icon in the upper left corner of any of the display regions 1001-1004. For example, FIG. 10B shows an example GUI 1010 that can be displayed in response to clicking the arrow icon in the upper left corner of the first display region 1001 of FIG. 10A.

Each of the display regions 1001-1003 includes a respective slice slider on the right-hand side (similar to the slice slider 618 of FIG. 6) that can be used to adjust the slice or cross-section displayed in that particular display region (such as described with reference to FIGS. 5 and 6). Instead of a reticle, object selection is performed by aligning a pair of orthogonal lines (or crosshair) 1012 and 1014 with the position of the desired object. For example, the position of the target may be marked by the intersection of the lines 1012 and 1014. In some implementations, a user may adjust the crosshair in any of the display regions 1001-1003 and the GUI 1000 may change the positions of the crosshair in the other display regions based on the user adjustment.

The display region 1004 may be substantially similar to the volumetric display region 630 of FIG. 6. In some implementations, the GUI 1000 may project any one of the cross-sectional views in the display regions 1001-1003 onto the 3D model of the anatomy in the display region 1004. For example, the cross-sectional view in an active display region, at any given time, may be projected onto the 3D model. As used herein, the term “active display region” may refer any of the display regions 1001-1003 that the user has selected, highlighted, or is currently interacting with. Example suitable interacts include panning, zooming, or adjusting a brightness or contrast of the image, among other examples. In the example of FIGS. 10A and 10B, such interactions may be provided via the toolbar 1005.

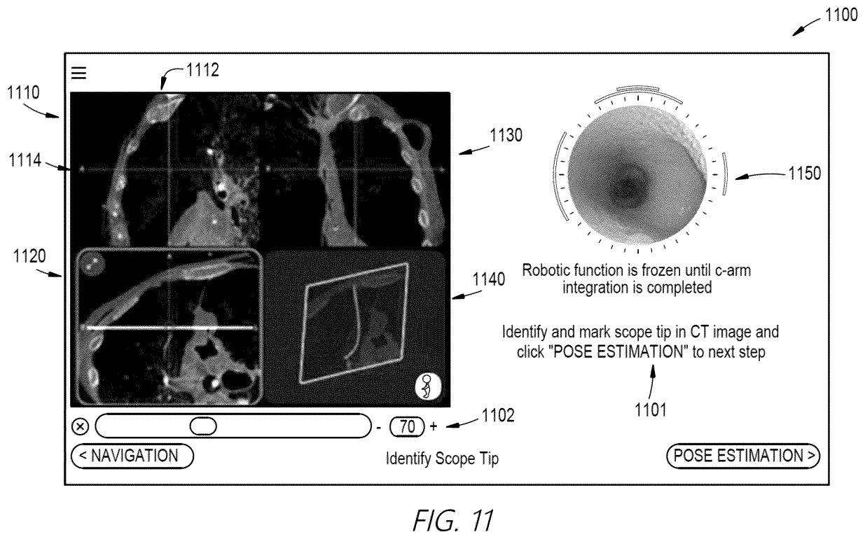

FIG. 11 shows another example GUI 1100 usable for identifying objects in an anatomy, according to some implementations. In some implementations, the GUI 1100 may be one example of the GUI 505 of FIG. 5. With reference for example to FIG. 5, the GUI 1100 may be generated by the system 500 based at least in part on tomographic image data (such as the image data 512).

The GUI 1100 includes 5 display regions 1110-1150 and instructions 1101 to a user for selecting an object. The display regions 1110-1140 are substantially similar to the display regions 1001-1004, respectively, of FIG. 10A. With reference for example to FIG. 5, each of the first three display regions 1110-1130 may display one of the cross-sectional views 501-503 and the fourth display region 1140 may display the volumetric view 504The GUI 1100 also includes a camera display region 1150. In some implementations, the camera display region 1150 may be substantially similar, if not identical, to the camera display region 640 of FIG. 6. With reference for example to FIG. 5, the camera display region 1150 may display real-time images (or video) of the anatomy based on the camera data 514. The display region 1140 may be substantially similar to the volumetric display region 630 of FIG. 6. In some implementations, the display region 1140 may display a virtual plane indicating a spatial relationship between the 3D model of the anatomy and the cross-sectional view depicted by an active display region.

A user may select any one of the display regions 1110-1130 to be an active display region at a given time. As shown in FIG. 11, the display region 1120 is currently highlighted as the active display region. In some implementations, a user may expand the active display region by tapping or clicking the arrow icon in the upper left corner of any of the active display region. In the example of FIG. 11, the arrow icon is only shown in the active display region. The GUI 1100 further includes a slice slider 1102 that can be used to adjust the slice or cross-section displayed in the active display region (such as described with reference to FIGS. 5 and 6). In the example of FIG. 11, the slice slider 1102 also includes a slice number or identifier (shown as “70” in FIG. 11) indicating which slice is currently being displayed in the active display region. Object selection is performed by aligning a pair of orthogonal lines (or crosshair) 1112 and 1114 with the position of the desired object (such as described with reference to FIG. 10A). In some implementations, a user may adjust the crosshair in the active display region and the GUI 1100 may change the positions of the crosshair in the other display regions based on the user adjustment.

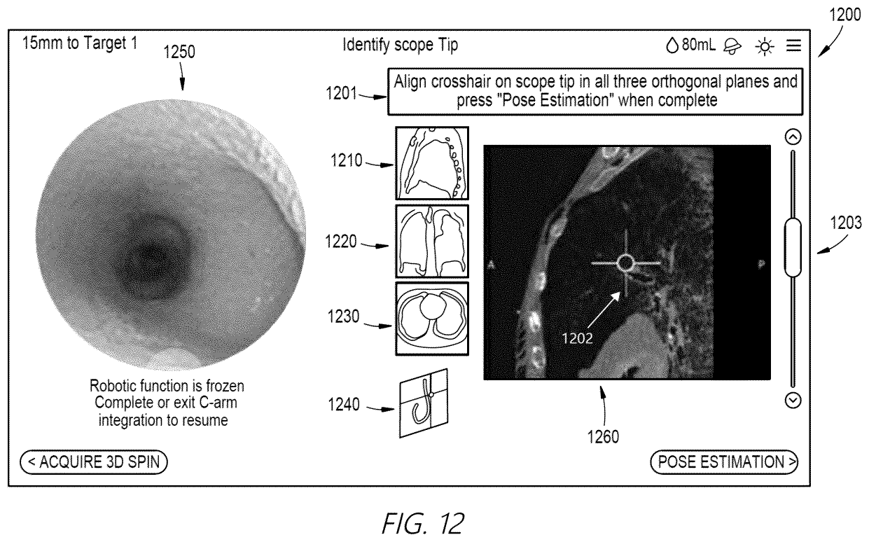

FIG. 12 shows another example GUI 1200 usable for identifying objects in an anatomy, according to some implementations. In some implementations, the GUI 1200 may be one example of the GUI 505 of FIG. 5. With reference for example to FIG. 5, the GUI 1200 may be generated by the system 500 based at least in part on tomographic image data (such as the image data 512).

The GUI 1200 includes 6 display regions 1210-1260 and instructions 1201 to a user for selecting an object. The display regions 1210-1240 are substantially similar to the display regions 1110-1140, respectively, of FIG. 11. With reference for example to FIG. 5, each of the first three display regions 1210-1230 may display one of the cross-sectional views 501-503 and the fourth display region 1240 may display the volumetric view 504. The GUI 1200 further includes a camera display region 1250, which may be substantially similar, if not identical, to the camera display regions 640 and 1140 of FIGS. 6 and 11, respectively. With reference for example to FIG. 5, the camera display region 1250 may display real-time images (or video) of the anatomy based on the camera data 514.

A user may select any one of the display regions 1210-1230 to be an active display region at a given time. As shown in FIG. 12, the display region 1210 is currently highlighted as the active display region. In some implementations, the GUI 1200 may display an expanded view of the active display region 1210 in the display region 1260. The GUI 1200 further includes a slice slider 1203 that can be used to adjust the slice or cross-section displayed in the display region 1260 (such as described with reference to FIGS. 5 and 6). The display region 1260 further includes a reticle 1202 that can be used to select the desired object. In some implementations, the reticle 1202 may be one example of any of the reticles 602 or 910 of FIGS. 6 and 9, respectively. The display region 1240 may be substantially similar to the volumetric display region 630 of FIG. 6. In some implementations, the GUI 1200 may project a virtual plane onto the 3D model of the anatomy in the display region 1240, where the virtual plane represents the cross-sectional view of the active display region.

The example GUIs described with reference to FIGS. 5-12 may be used to specify a position of an instrument within an anatomy. As described with reference to FIG. 4, some registration techniques may require knowledge of the position and orientation (or heading) of the instrument. In some implementations, the instrument orientation may be determined in a separate workflow step (such as after determining the instrument position). In some other implementations, a user may indicate the orientation (or heading) of an instrument using the same GUI for selecting the instrument position. For example, in addition to selecting the position of the instrument, the GUI may prompt the user to specify a 3D vector (or individual 2D vectors in each cross-sectional view) pointing in the instrument's direction of heading.

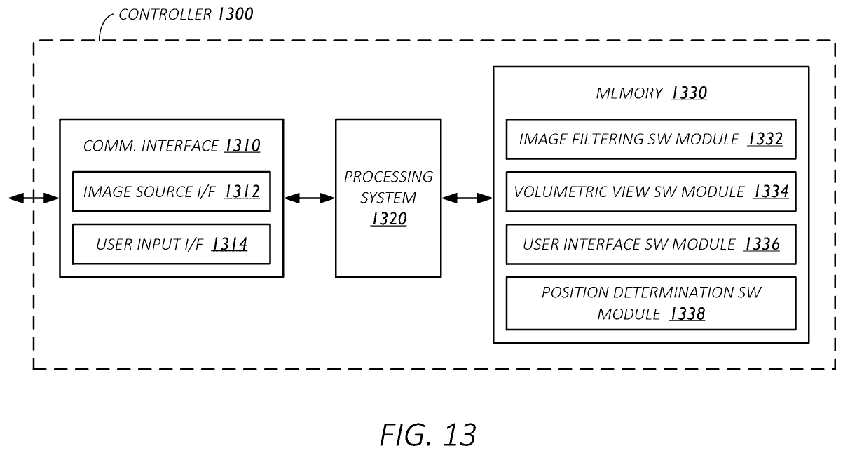

FIG. 13 shows a block diagram of an example controller 1300 for a medical system, according to some implementations. In some implementations, the controller 1300 may be one example of the system 500 of FIG. 5. More specifically, the controller 1300 is configured to determine a position of an object (such as an instrument or a target) within an anatomy based at least in part on image data received from an imaging system.

The controller 1300 includes a communication interface 1310, a processing system 1320, and a memory 1330. The communication interface 1310 is configured to communicate with one or more components of the medical system. More specifically, the communication interface 1310 includes an image source interface (I/F) 1312 for communicating with one or more image sources (such as the CT imaging system 310 and/or the fluoroscopy imaging system 312 of FIG. 3) and a user input interface (I/F) 1314 for communicating with one or more user input devices (such as any of the user input controls 214 and/or the I/O components 240 of FIG. 2). In some implementations, the image source I/F 1312 may receive image data representing a 3D model of the anatomy. In some implementations, the user input I/F 1314 may receive one or more user inputs associated with a graphical user interface (GUI).

The memory 1330 may include a non-transitory computer-readable medium (including one or more nonvolatile memory elements, such as EPROM, EEPROM, Flash memory, or a hard drive, among other examples) that may store the following software (SW) modules: an image filtering SW module 1332 to filter the image data based at least in part on one or more known properties of the object within the anatomy; a volumetric view SW module 1334 to generate a volumetric view of the anatomy based on the filtered image data so that the object is highlighted in the volumetric view; a user interface SW module 1336 to display the volumetric view in the GUI; and a position determination SW module 1338 to determine the position of the object within the anatomy based on the one or more user inputs. Each of the software modules 1332-1338 includes instructions that, when executed by the processing system 1320, causes the controller 1300 to perform the corresponding functions.

The processing system 1320 may include any suitable one or more processors capable of executing scripts or instructions of one or more software programs stored in the controller 1300 (such as in the memory 1330). For example, the processing system 1320 may execute the image filtering SW module 1332 to filter the image data based at least in part on one or more known properties of the object within the anatomy. The processing system 1320 also may execute the volumetric view SW module 1334 to generate a volumetric view of the anatomy based on the filtered image data so that the object is highlighted in the volumetric view. The processing system 1320 may execute the user interface SW module 1336 to display the volumetric view in the GUI. The processing system 1320 may further execute the position determination SW module 1338 to determine the position of the object within the anatomy based on the one or more user inputs.

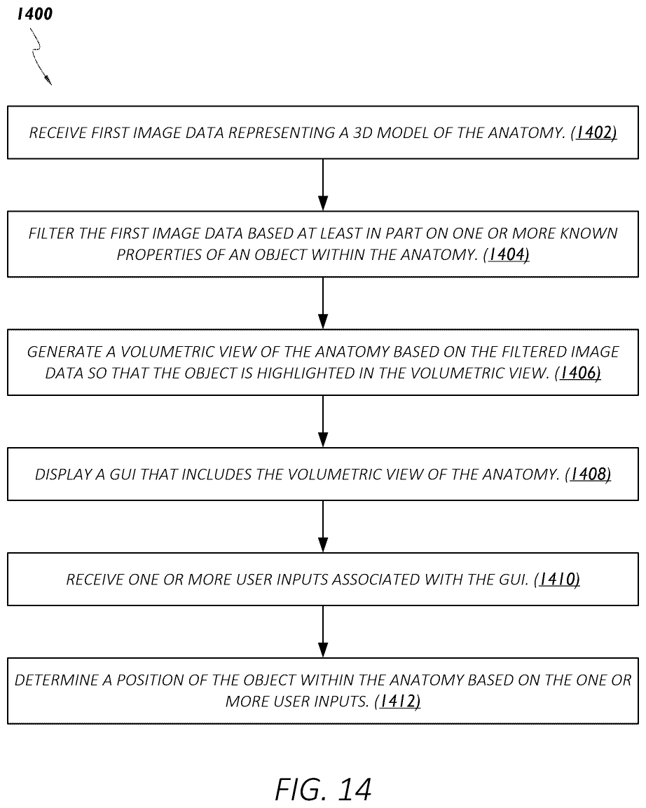

FIG. 14 shows an illustrative flowchart depicting an example operation 1400 for identifying objects in an anatomy, according to some implementations. In some implementations, the example operation 1400 may be performed by a controller for a medical system such as the controller 1300 of FIG. 13 or the system 500 of FIG. 5.

The controller may receive first image data representing a 3D model of the anatomy (1402). The controller may filter the first image data based at least in part on one or more known properties of an object within the anatomy (1404). The controller may generate a volumetric view of the anatomy based on the filtered image data so that the object is highlighted in the volumetric view (1406). The controller may display a GUI that includes the volumetric view of the anatomy (1408). The controller may receive one or more user inputs associated with the GUI (1410). The controller may further determine a position of the object within the anatomy based on the one or more user inputs (1412).

In some aspects, the object may be a medical instrument or a target for a medical procedure (such as a nodule). In some implementations, the one or more known properties of the object may include an expected image intensity distribution associated with the object. In such implementations, the filtering of the first image data may include hiding one or more voxels of the first image data having intensities below a threshold intensity associated with the expected image intensity distribution.

In some aspects, the GUI may further display a plurality of cross-sectional views of the 3D model in a plurality of display regions, respectively, including a primary display region and one or more thumbnail display regions. In some implementations, the GUI may further include a feature for exchanging the cross-sectional view displayed in the primary display region with any of the cross-sectional views displayed in the one or more thumbnail display regions based on the one or more user inputs.

In some implementations, the GUI may further include a zoom feature for adjusting a magnification of the plurality of cross-sectional views, a contrast feature for adjusting a contrast of the plurality of cross-sectional views, a brightness feature for adjusting a brightness of the plurality of cross-sectional views, or a slice feature for changing a depth of a cross-sectional plane depicted by the cross-sectional view displayed in the primary display region.

In some aspects, the GUI may further include a reticle for selecting the position of the object in relation to the anatomy based on the one or more user inputs, where the reticle is displayed as an interactive feature in the primary display region and as a static feature in each of the one or more thumbnail display regions. In such aspects, the controller may display a new cross-sectional view of the 3D model in at least one of the one or more thumbnail display regions responsive to movement of the reticle in the primary display region. In some implementations, the controller may further estimate a pose of the object based on a position of the reticle.

In some aspects, the volumetric view may include a virtual plane intersecting the anatomy at a cross-section depicted by the cross-sectional view displayed in the primary display region, where the virtual plane indicates a position of the reticle in relation to the volumetric view. In some implementations, the one or more user inputs may include a selection input associated with the volumetric view. In such implementations, the controller may further align the reticle with the anatomy in each of the plurality of display regions based on the selection input associated with the volumetric view.

Those of skill in the art will appreciate that information and signals may be represented using any of a variety of different technologies and techniques. For example, data, instructions, commands, information, signals, bits, symbols, and chips that may be referenced throughout the above description may be represented by voltages, currents, electromagnetic waves, magnetic fields or particles, optical fields or particles, or any combination thereof.

The various illustrative logics, logical blocks, modules, circuits and algorithm processes described in connection with the implementations disclosed herein may be implemented as electronic hardware, computer software, or combinations of both. The interchangeability of hardware and software has been described generally, in terms of functionality, and illustrated in the various illustrative components, blocks, modules, circuits and processes described herein. Whether such functionality is implemented in hardware or software depends upon the particular application and design constraints imposed on the overall system.

In the foregoing specification, implementations have been described with reference to specific examples thereof. It will, however, be evident that various modifications and changes may be made thereto without departing from the broader scope of the disclosure as set forth in the appended claims. The specification and drawings are, accordingly, to be regarded in an illustrative sense rather than a restrictive sense.

As used herein, a phrase referring to “at least one of” a list of items refers to any combination of those items, including single members. As an example, “at least one of: a, b, or c” is intended to cover: a, b, c, a-b, a-c, b-c, and a-b-c.

Various modifications to the implementations described in this disclosure may be readily apparent to those skilled in the art, and the generic principles defined herein may be applied to other implementations without departing from the spirit or scope of this disclosure. Thus, the claims are not intended to be limited to the implementations shown herein, but are to be accorded the widest scope consistent with this disclosure, the principles and the novel features disclosed herein.

What is claimed is:

1. A method for identifying objects in an anatomy, comprising:
receiving first image data representing a three-dimensional (3D) model of the anatomy;
filtering the first image data based at least in part on one or more known properties of an object within the anatomy;
generating a volumetric view of the anatomy based on the filtered image data so that the object is highlighted in the volumetric view;
displaying a graphical user interface (GUI) that includes the volumetric view of the anatomy;
receiving one or more user inputs associated with the GUI; and
determining a position of the object within the anatomy based on the one or more user inputs.

2. The method of claim 1, wherein the object is a medical instrument or a target for a medical procedure.

3. The method of claim 1, wherein the GUI further displays a plurality of cross-sectional views of the 3D model in a plurality of display regions, respectively, including a primary display region and one or more thumbnail display regions.

4. The method of claim 3, wherein the GUI further includes a feature for exchanging the cross-sectional view displayed in the primary display region with any of the cross-sectional views displayed in the one or more thumbnail display regions based on the one or more user inputs.

5. The method of claim 3, wherein the GUI further includes a zoom feature for adjusting a magnification of the plurality of cross-sectional views, a contrast feature for adjusting a contrast of the plurality of cross-sectional views, a brightness feature for adjusting a brightness of the plurality of cross-sectional views, or a slice feature for changing a depth of a cross-sectional plane depicted by the cross-sectional view displayed in the primary display region.

6. The method of claim 3, wherein the GUI further includes a reticle for selecting the position of the object in relation to the anatomy based on the one or more user inputs, the reticle being displayed as an interactive feature in the primary display region and as a static feature in each of the one or more thumbnail display regions, the method further comprising:
displaying a new cross-sectional view of the 3D model in at least one of the one or more thumbnail display regions responsive to movement of the reticle in the primary display region.

7. The method of claim 6, wherein the volumetric view further includes a virtual plane intersecting the anatomy at a cross-section depicted by the cross-sectional view displayed in the primary display region, the virtual plane indicating a position of the reticle in relation to the volumetric view.

8. The method of claim 6, wherein the one or more user inputs include a selection input associated with the volumetric view, the method further comprising:
aligning the reticle with the anatomy in each of the plurality of display regions based on the selection input associated with the volumetric view.

9. The method of claim 6, further comprising:
estimating a pose of the object based on a position of the reticle.

10. The method of claim 1, wherein the one or more known properties of the object include an expected image intensity distribution associated with the object, the filtering of the first image data comprising:
hiding one or more voxels of the first image data having intensities below a threshold intensity associated with the expected image intensity distribution.

11. A controller for a medical system, comprising:
a processing system;
a memory storing instructions that, when executed by the processing system, cause the controller to:
receive first image data representing a three-dimensional (3D) model of an anatomy;
filter the first image data based at least in part on one or more known properties of an object within the anatomy;
generate a volumetric view of the anatomy based on the filtered image data so that the object is highlighted in the volumetric view;
display a graphical user interface (GUI) that includes the volumetric view of the anatomy;
receive one or more user inputs associated with the GUI; and
determine a position of the object within the anatomy based on the one or more user inputs.

12. The controller of claim 11, wherein the object is a medical instrument or a target for a medical procedure.

13. The controller of claim 11, wherein the GUI further displays a plurality of cross-sectional views of the 3D model in a plurality of display regions, respectively, including a primary display region and one or more thumbnail display regions.

14. The controller of claim 13, wherein the GUI further includes a feature for exchanging the cross-sectional view displayed in the primary display region with any of the cross-sectional views displayed in the one or more thumbnail display regions based on the one or more user inputs.

15. The controller of claim 13, wherein the GUI further includes a zoom feature for adjusting a magnification of the plurality of cross-sectional views, a contrast feature for adjusting a contrast of the plurality of cross-sectional views, a brightness feature for adjusting a brightness of the plurality of cross-sectional views, or a slice feature for changing a depth of a cross-sectional plane depicted by the cross-sectional view displayed in the primary display region.

16. The controller of claim 13, wherein the GUI further includes a reticle for selecting the position of the object in relation to the anatomy based on the one or more user inputs, the reticle being displayed as an interactive feature in the primary display region and as a static feature in each of the one or more thumbnail display regions, execution of the instructions further causing the controller to:
display a new cross-sectional view of the 3D model in at least one of the one or more thumbnail display regions responsive to movement of the reticle in the primary display region.

17. The controller of claim 16, wherein the volumetric view further includes a virtual plane intersecting the anatomy at a cross-section depicted by the cross-sectional view displayed in the primary display region, the virtual plane indicating a position of the reticle in relation to the volumetric view.

18. The controller of claim 16, wherein the one or more user inputs include a selection input associated with the volumetric view, execution of the instructions further causing the controller to:
align the reticle with the anatomy in each of the plurality of display regions based on the selection input associated with the volumetric view.

19. The controller of claim 16, wherein execution of the instructions further causes the controller to:
estimate a pose of the object based on a position of the reticle.

20. The controller of claim 11, wherein the one or more known properties of the object include an expected image intensity distribution associated with the object, the filtering of the first image data comprising:
hiding one or more voxels of the first image data having intensities below a threshold intensity associated with the expected image intensity distribution.
